(19) United States
(12) Patent Application Publication
(10) Pub. No.: US 2024/0424494 A1
(43) Pub. Date: Dec. 26, 2024

(54) HIGH EFFICIENCY MICROFLUIDIC BIOBARRIER PLATFORM, SYSTEM, AND METHOD

(71) Applicant: Rutgers, The State University of New Jersey, New Brunswick, NJ (US)

(72) Inventors: Juan Sebastian Pena-Carvajal, New Brunswick, NJ (US); Maribel Vazquez, New Brunswick, NJ (US)

(73) Assignee: Rutgers, The State University of New Jersey, New Brunswick, NJ (US)

(21) Appl. No.: 18/618,677

(22) Filed: Mar. 27, 2024

Related U.S. Application Data
(60) Provisional application No. 63/521,975, filed on Jun. 20, 2023.
(60) Provisional application No. 63/520,813, filed on Aug. 21, 2023.

(51) Int. Cl. B01L 3/00; G01N 33/50

(52) U.S. Cl. CPC B01L 3/502761; G01N 33/5008; B01L 2300/0645; B01L 2300/0861; B01L 2400/0472

(57) ABSTRACT

A high efficiency microfluidic biobarrier platform, system, and method permit an in vitro examination of a biobarrier under physiological flow conditions. The platform comprises an inlet, a first member, and an outlet. The inlet receives an influx of fluid. The first member retains the biobarrier, and the member includes a channel in fluid communication with the inlet to receive the fluid and to pass the fluid adjacent to the biobarrier. The outlet directs an outflux of the fluid. The microfluidic platform features built-in plastic electrodes to assess changes in biobarrier impedance in real-time. A system includes the platform, a pump to provide the fluid, a media collecting reservoir to receive the fluid from the platform, and an impedance analyzer to measure changes in biobarrier impedance. A method uses the platform in the system to determine the integrity of the biobarrier and its permeability across the cell barrier.

### CROSS-REFERENCE TO RELATED APPLICATION

This application claims priority to pending U.S. provisional application no. U.S. 63/521,975, filed Jun. 20, 2023, and pending U.S. provisional application no. U.S. 63/520,813, filed Aug. 21, 2023, the disclosures of which are incorporated herein by reference in their entireties.

### FIELD OF THE DISCLOSURE

The present disclosure relates generally to biobarriers, and, more particularly, to a high efficiency microfluidic bio-barrier (HμB) platform, system, and method.

### BACKGROUND OF THE DISCLOSURE

Understanding the transport of solutes and the integrity of membranes across human biobarriers is essential for the development and evaluation of medications against disease. Biobarriers, which refer to cellular monolayers regulating molecule transport within organs and tissues are ubiquitous in the human body. Examples include the blood brain barrier (BBB), the pulmonary epithelial barrier, and the glomerular filter barrier, all of which play a critical role in regulating the homeostasis of organs and tissues. However, the breakdown of these barriers results in progressive diseases such as Alzheimer's diseases (AD), chronic cystic fibrosis, and kidney disease, which do not have a cure. Hence, novel therapies are needed to devise solutions against these diseases. Nonetheless, the process of testing multiple drugs and assessing their efficacy is a slow and costly process that often brings promising therapies to halt. Furthermore, the majority of drug development practices in the pharmaceutical and cosmetic industry have been subjected to scrutiny due to their increasing use of animals for experimental testing. In response, the new Modernization Act 2.0, passed by the Food and Drug Administration (FDA) in December 2022, has supported the development of alternative testing methods that do not need to use animals before clinical trials. This new act has propelled the development of new engineering approaches to understand the physiological parameters of biobarriers and optimize the costly process of drug manufacturing. Novel technology such as microfluidics aim to replicate the geometry and physiological conditions of biobarriers in vitro to understand the empirical mechanisms that drive the onset and progression of disease, so new therapies can be developed to restore function. Particularly, microfluidics have become a popular alternative in the pharmaceutical industry to model biobarriers to test the efficacy of pharmacological agents without the use of animals.

Vascular biobarriers such as the BBB and the inner blood retinal barrier (iBRB) selectively control metabolite exchange with the brain and retina, respectively. As shown in FIG. 1, in the prior art, an in vivo IBRB 10 can be modeled in vitro 12, with a lumen 14 and an interstitial space 16 oriented and positioned for testing of medicines.

There is a need to develop novel drug testing platforms that can yield high throughput results in vitro. For example, multiple drugs are being designed and tested against diabetic retinopathy which affects the integrity of the iBRB. However, in the prior art, it has been difficult to examine a biobarrier in vitro under physiological flow conditions to test such drugs. The challenge of in vitro models is that such models are mostly static, which does not recapitulate the physiological conditions of the body. In addition, the inclusion of different metrics to assess barrier integrity has been challenging, as most models only feature a membrane interface and can only measure barrier permeability.

In another example, the testing of cosmetics on the skin can involve the testing of biobarriers, such as the skin barrier to test the efficacy of ingredients in the skin, while minimizing a systemic reaction. Yet, thorough testing of the effects of cosmetics on the skin barrier has been scrutinized due to ethical concerns, such as for the health and well-being of human or animal test subjects during in vivo testing. Hence, there is a need to move away from in vivo testing of medicines and cosmetics to a more in vitro setting.

### SUMMARY OF THE DISCLOSURE

According to an embodiment consistent with the present disclosure, a high efficiency microfluidic bio-barrier (HμB) platform, system, and method permit an in vitro examination of a biobarrier under physiological flow conditions.

In an embodiment, a platform comprises an inlet, a first member, and an outlet. The first member can be a component in the shape of a cuboid. Alternatively, the first member can have other shapes and sizes. The first member can be a top cover of the platform. Alternatively, the first member can be a bottom cover of the platform. The first member is configured to retain a biobarrier. The inlet is configured to receive an influx of fluid. The outlet is configured to direct an outflux of the fluid. The inlet can include two separate inlets, and the outlet can include two separate outlets. The separate inlets and outlets can be arranged to have different flow rates of the fluid along each side of the platform. The platform can be used to model capillary blood flow and interstitial flow, to model different flow rates on either side of the biobarrier.

The first member includes a channel in fluid communication with the inlet to receive the fluid and to pass the fluid adjacent to the biobarrier. The first member can include a window adjacent to the biobarrier and configured to permit visual observation of the biobarrier to determine a visual property of the biobarrier. The window can be an empty aperture. Alternatively, the window can include an observation member selected from the group consisting of: a transparent member and a translucent member.

The platform can further comprise a second member disposed adjacent to the channel and being electrically conductive, and the second member is configured to permit an electrical measurement of the biobarrier to determine an electrical property of the biobarrier. The electrical property of the biobarrier can be selected from the group consisting of: an electrical capacitance, an electrical impedance, an electrical voltage, and an electrical resistivity. The second member can be a component in the shape of a cuboid. Alternatively, the second member can have other shapes and sizes.

In another embodiment, a system comprises a pump, a media collecting reservoir, and a platform. The pump is configured to drive fluid into the platform. The pump can be configured to deliver specific flow rates of the fluid, with the specific flow rates being at a physiological level. The media collecting reservoir is configured to receive the fluid. The platform includes an inlet, a first member, and an outlet. The inlet is connected to the pump and is configured to receive an influx of the fluid. The first member is configured to retain a biobarrier, and the first member includes a channel in fluid communication with the inlet to receive the fluid and to pass the fluid adjacent to the biobarrier. The outlet is connected to the media collecting reservoir and configured to direct an outflux of the fluid to the media collecting reservoir.

The system can include a solute concentration measuring device configured to measure a concentration of solutes in the outflux of the fluid, thereby measuring a permeability across or associated with the biobarrier. The system can further comprises a visual observation device adjacent to the platform, wherein the first member can include a window adjacent to the biobarrier, wherein the visual observation device can be disposed adjacent to the window, and wherein the first member can be configured to permit visual observation of the biobarrier by the visual observation device through the window to determine a visual property of the biobarrier. The window can be an empty aperture. Alternatively, the window can include an observation member selected from the group consisting of: a transparent member and a translucent member. Still further, the visual observation device can be selected from the group consisting of a microscope, a lens, and a camera.

The system can further comprise an electrical analyzer configured to determine an electrical property of the biobarrier. The platform can include a second member disposed adjacent to the channel and being electrically conductive. The second member can be electrically connected to the electrical analyzer to permit an electrical measurement of the biobarrier to determine the electrical property of the biobarrier. The second member can be an electrode. The electrical property of the biobarrier can be selected from the group consisting of: an electrical capacitance, an electrical impedance, an electrical voltage, and an electrical resistivity. The electrical analyzer can be an impedance analyzer. The second member can be connected to the electrical analyzer via a conductive device, such as a conductive wire or a conductive tape. The conductive wire can be a copper wire. The conductive tape can be a copper tape. The conductive wire or tape can be bonded to a conductive side of the second member. A portion of the conductive wire or tape sticks out of the platform, and can be connected to the electrical analyzer.

In a further embodiment, a method comprises providing a platform including a channel therethrough, positioning a biobarrier in a platform and adjacent to the channel, passing a fluid through the channel to be adjacent to the biobarrier, and determining a property of the biobarrier relative to the fluid. The method can comprise the platform or device with two independent fluid channels along a biobarrier membrane, with each of the two fluid channels having an inlet and an outlet. Each inlet features a corresponding separate outlet, connecting only at the biobarrier membrane where fluid can flow across.

The platform can include a window adjacent to the biobarrier. The determining of the property can includes visually observing the biobarrier through the window to determine a visual property of the biobarrier. The property can include a permeability of the biobarrier. The platform can include an electrode, and the determining of the property can include measuring an electrical property of the biobarrier using the electrode. The electrical property of the biobarrier can be selected from the group consisting of: an electrical capacitance, an electrical impedance, an electrical voltage, and an electrical resistivity. The platform and the system and method using the platform efficiently streamline the testing of drugs that could cross a BBB. The platform, system, and method can mimic an iBRB, for example, by incorporating physiological flow rates, such as capillary flow rate and interstitial flow rate. Such flow rates more closely mimic the in vivo environment.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a shift away from an in vivo IBRB to an in vitro IBRB in biobarrier examination in the prior art.

FIG. 2 illustrates a top plan view of the high efficiency microfluidic bio-barrier (HμB) platform of an embodiment.

FIG. 3 illustrates an exploded top view of components of the HμB platform of FIG. 2.

FIG. 4 illustrates a side elevational view of the HμB platform of FIG. 2.

FIG. 5A illustrates a side cross-sectional view of the biobarrier positioned in the HμB platform of FIG. 2.

FIG. 5B illustrates an alternative side cross-sectional view of the biobarrier positioned in the HμB platform of FIG. 2.

FIG. 6 illustrates a top plan view of the HμB platform with inlets and outlets showing the flow of fluids through the HμB platform.

FIG. 7 illustrates a side elevational view of the HμB platform of FIG. 6.

FIG. 8 illustrates a side cross-sectional view of the biobarrier with the flow of fluids in FIG. 6.

FIG. 9 illustrates a top plan view of the HμB platform of FIG. 2 without leads.

FIG. 10 illustrates a side exploded view of the HμB platform of FIG. 9.

FIG. 11 illustrates a side cross-sectional view of the HμB platform of FIG. 9.

FIG. 12 illustrates a top front view of a system having the HμB platform.

FIG. 13 illustrates a top perspective view of the HμB platform in the system of FIG. 12.

FIGS. 14A-14F illustrate various views of cultures of Muller glia in the HμB platform.

FIG. 15 illustrates a graph of a magnitude of a current opposed by the resistance and capacitance of Muller glia monolayers.

FIG. 16 illustrates a graph of a phase diagram of Muller glia monolayer impedance.

FIG. 17 illustrates a graph of normalized impedance of Muller glia and endothelial cells.

FIG. 18 illustrates a graph of normalized impedance of endothelial cells.

FIG. 19A illustrates a three-dimensional computer simulation of the flow along and around the biobarrier in the HμB platform.

FIG. 19B illustrates a computer model of fluid velocity in the microchannels of the HμB platform and across the biobarrier.

FIG. 19C illustrates a computer model of a velocity profile across each microchannel.

FIG. 19D illustrates a computer model of a shear stress profile across the biobarrier.

FIG. 20 is a flowchart of operation of the HμB platform.

FIG. 21A illustrates a top view of an alternative embodiment of the HμB platform shown in FIG. 2.

FIG. 21B illustrates a top front side perspective view of the HμB platform shown in FIG. 21A with parts separated.

FIG. 21C illustrates a side view of the HμB platform shown in FIG. 21A.

FIG. 22A illustrates fluid flows into and out of the HμB platform shown in FIG. 21A.

FIG. 22B illustrates a top view of the membrane positioned in the HμB platform with flows above and below the membrane.

FIG. 22C illustrates a top view of a top layer of cells above the membrane positioned in the HμB platform with fluid flows above and below the top layer of cells.

FIG. 22D illustrates a bottom view of a bottom layer of cells below the membrane positioned in the HμB platform with fluid flows above and below the bottom layer of cells.

FIG. 23 illustrates a table of definitions of physical properties.

FIG. 24A illustrates a top view of acrylic sheets.

FIG. 24B illustrates a top view of laser-cut layers of the HμB platform.

FIG. 24C illustrates a top view of a set of components of the HμB platform.

FIG. 24D illustrates a top view of the stack of components in FIG. 24C assembled as the HμB platform.

FIG. 24E illustrates a top front view of a heat press used to press all of the components of the HμB platform together.

FIG. 25 illustrates a sequence of steps for seeding the cells in the HμB platform.

FIG. 26A illustrates a top front side perspective view of an incubator chamber retaining a set of HμB platforms

FIG. 26B illustrates a top front side perspective view of the incubator chamber of FIG. 26A with parts separated.

FIG. 27 illustrates an alternative embodiment of the system in FIG. 12 having the HμB platform positioned therein.

FIG. 28A illustrates a top front perspective view of fluid flows in the HμB platform.

FIG. 28B illustrates a bottom front perspective view of fluid flows in the HμB platform.

FIG. 28C illustrates a top side perspective view of fluid flows in the HμB platform.

FIG. 29A illustrates a top plan view of fluid flows in the HμB platform.

FIG. 29B illustrates another top front perspective view of fluid flows in the HμB platform.

FIG. 29C illustrates a two-dimensional graphical representation of a side cross-sectional view of the HμB platform with velocity magnitudes of the fluid flows in the channels and across the membrane.

FIG. 29D illustrates an enlarged portion of the graphical representation of the membrane in FIG. 29C.

FIG. 29E illustrates a graph of the velocity magnitudes of the fluid flows in FIG. 29C.

FIGS. 30A-30C illustrate mass transport concentration gradients across the channels in the HμB platform for Rhodamine B, 70 kDa dextran, and 150 kDa dextran, respectively.

FIGS. 30D-30F illustrate graphs of concentration gradients across the channels over time in the HμB platform for Rhodamine B, 70 kDa dextran, and 150 kDa dextran, respectively.

FIGS. 30G-30I illustrate graphs of normalized concentration of solutes over time at the top and bottom outlet of the HμB platform for Rhodamine B, 70 kDa dextran, and 150 kDa dextran, respectively.

FIGS. 31A-31C illustrate brightfield images of confluent cell barriers under flow for endothelial cells (ECs), Muller glia (MG), and a combination of ECs and MGs, respectively.

FIGS. 31A-31C illustrate brightfield images of confluent cell barriers under flow for endothelial cells (ECs), Muller glia (MG), and a combination of ECs and MGs, respectively.

FIGS. 31D-31F illustrate enlarged portions of the brightfield images of confluent cell barriers under flow shown in FIGS. 31A-31C, respectively.

FIGS. 31G-31I illustrate brightfield images of confluent cell barriers under flow for diabetic endothelial cells (DECs), diabetic Muller glia (DMG), and a combination of DECs and DMGs, respectively.

FIGS. 31J-31L illustrate enlarged portions of the brightfield images of confluent cell barriers under flow shown in FIGS. 31G-31I, respectively.

FIG. 32A illustrates a brightfield image of a confluent combination cell barrier with ECs on the top of the membrane and MG on the bottom side of the membrane.

FIG. 32B illustrates an enlarged portion of the image shown in FIG. 32A.

FIG. 33 illustrates a graph of the impedance of the fluids in the HμB platform.

FIG. 34A illustrates a brightfield image of a healthy confluent cell barrier.

FIG. 34B illustrates a brightfield image of an unhealthy confluent cell barrier.

FIG. 34C illustrates a graph of an impedance comparison of the healthy cell barrier from FIG. 34A vs. the unhealthy cell barrier from FIG. 34B.

FIG. 34D illustrates a graph of impedance over time of the healthy cell barrier from FIG. 34A.

FIG. 35 illustrates a graph of impedance of healthy and diabetic cell barriers in the HμB platform over time.

FIG. 36 illustrates a graph of permeability of cell barriers in the HμB platform over time.

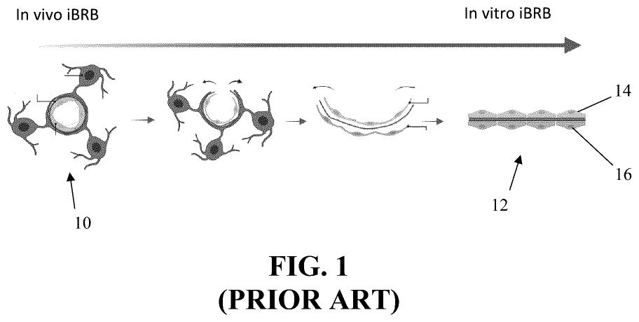

It is noted that the drawings are illustrative and are not necessarily to scale.

### DETAILED DESCRIPTION OF CERTAIN EMBODIMENTS OF THE DISCLOSURE

Example embodiments consistent with the teachings included in the present disclosure are directed to a high efficiency microfluidic bio-barrier (HμB) platform 100, a system 200, and a method 300 which permit an in vitro examination of a biobarrier 12 such as a membrane or tissue under physiological flow conditions in real-time. For example, the (HμB) platform 100, the system 200, and the method 300 can permit an in vitro examination of an integrity of the biobarrier 12 which can be measured in real-time. The biobarrier 12 can be a cell monolayer or tissue which can be examined under physiological flow conditions. For example, the biobarrier 12 can be a BBB, a gut barrier, a lung barrier, or any known biological barrier or membrane.

The HμB platform 100 implements a microfluidic system which replicates the close cell proximity of any biobarrier of the body under physiological flow conditions. Accordingly, the HμB platform 100 having the biobarrier 12 can serve as a model of any biobarrier of the body to study cell transport of solutes and membrane integrity over time. The HμB platform 100 is adapted for use as a drug testing platform for pharmacokinetics, for use in disease modeling, and for use in evaluating cosmetics in an in vitro environment. The HμB platform 100 is constructed to be a standalone unit that can be easily manufactured and distributed and is configured to work in a plug-and-play manner in that the HμB platform 100 can be removed from (sterile) packaging and then prepared with a culture and easily connected to fluid sources that pass through the platform during use.

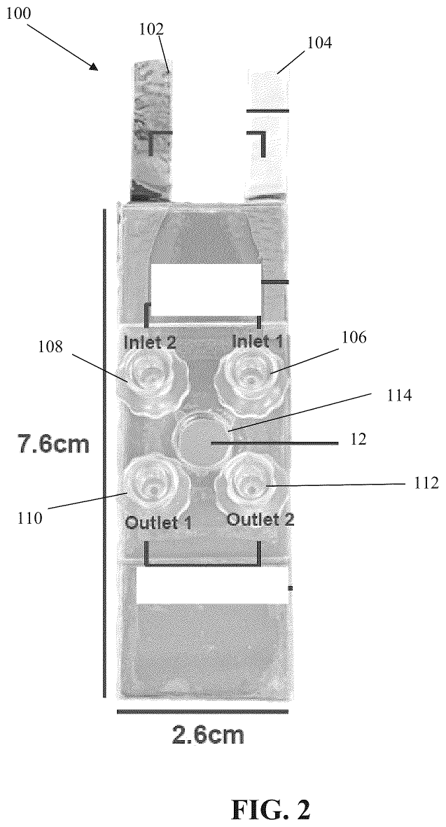

Referring to FIG. 2, the high efficiency microfluidic bio-barrier (HμB) platform 100 having the components described below is configured to provide a flow of fluid on, above, below, and across a biobarrier 12 to be tested and monitored. It is to be understood that the (HμB) platform 100 having the components described below can also be configured to provide a flow of gas on, above, below, and across a biobarrier 12 to be tested and monitored. The fluid can include any known elements and molecules, such as water, emulsions, pharmacological agents, fluorescent molecules, and any other known substances, including glucose, and pharmacological agents, to test and monitor the reaction of the biobarrier 12 to such substances. Such reactions can include visual changes of appearance, changes in electrical conductivity, changes in voltage, changes in resistivity, and changes in permeability of the biobarrier 12.

The biobarrier 12 can be a biological membrane or a tissue including a plurality of biological cells. In an example embodiment, the biobarrier 12 is an in vitro IBRB having a lumen 14 and an interstitial space 16. The HμB platform 100 has a plurality of conductive leads 102, 104 connected to electrodes described below. For example, the leads 102, 104 can be composed of copper. Alternatively, other known conductive materials can be used for the plurality of conductive leads 102, 104. For example, the conductive leads 102, 104 can be connected to electrically-conductive plastic electrodes, such as clear plastic electrodes, capable of measuring cell resistance, impedance, voltage, electrical current, and capacitance. It will be appreciated that other types of electrodes can be used, such as glass electrodes. In the illustrated embodiment, the conductive leads 102, 103 are located at the same end of the HμB platform 100 and protrude outwardly therefrom.

The HμB platform 100 also includes a plurality of inlets (inlet ports) 106, 108, each of which can be coupled to a tube or pipe configured to receive the fluid into a fluid passage in the HμB platform 100. The HμB platform 100 also includes a plurality of outlets (outlet ports) 110, 112, each of which can be coupled to respective tubing, such as a tube or pipe, configured to convey the fluid out of the fluid passage of the HμB platform 100. The inlets 106, 108 and the outlets 110, 112 can include, for example, Luer-locks configured to attach to the respective tubing (conduits). In an alternative embodiment, each of the inlets 106, 108 and the outlets 110, 112 can include any known attachment mechanism configured to attach the inlets 106, 108 and the outlets 110, 112 to the respective tubing (conduits). As shown, the inlets 106, 108 and the outlets 110, 112 are located along the same side of the HμB platform 100 to allow the user to easily connect (and disconnect) the HμB platform 100 to fluid sources. In particular, the inlets 106, 108 and the outlets 110, 112 are located along the top of the HμB platform 100 since the bottom is intended to be planar for placement of a flat surface, such as a microscope stage, etc. In the illustrated embodiment, the inlets 106, 108 are located side-by-side and the outlets 110, 112 are located side-by-side spaced from the inlets 106, 108.

The HμB platform 100 can be configured and dimensioned to be any length or width and can have different shapes, such as a rectangle as shown. For example, a longitudinal length of the platform 100 can be 7.6 cm, and a lateral width of the HμB platform 100 can be 2.6 cm. The HμB platform 100 can be configured and dimensioned to integrate within conventional microscope stages and biological devices. In other words, the HμB platform 100 can be shaped and sized to be received within a recess of a microscope stage.

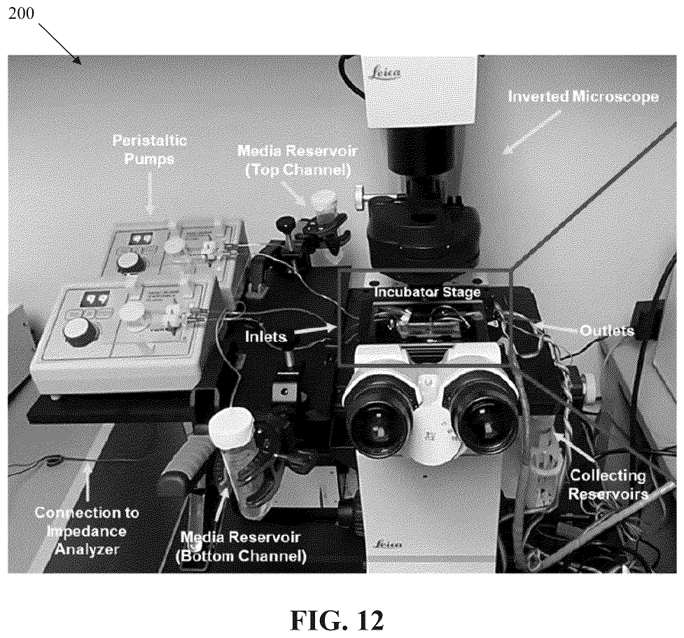

The HμB platform 100 further includes a window 114 through which the biobarrier 12 is visible. The window 114 can be an empty aperture permitting visual observation of the biobarrier 12. Alternatively, the window 114 can include a transparent or translucent member permitting visual observation of the biobarrier 12. In another alternative embodiment, the transparent or translucent member can be a lens configured to enlarge the visual appearance of the biobarrier 12. In addition, a visual observation device, such as a microscope as shown in FIG. 12 and described below, can be positioned adjacent to the window 114 to capture images of the biobarrier 12 through the window 114. The diameter of the window 114 can be, for example, 0.78 cm. The biobarrier can have a thickness of, for example, 10 μm, and a membrane porosity of 0.4 μm. In another example, the membrane porosity can be altered, depending on the biobarrier 12 or membrane that is used in the HμB platform 100 or device. A larger porosity, such as a porosity greater than 8 μm, can be used to model cell migration. Such altering of the membrane porosity can be key in modeling any kind of inflammation in the body, where leukocytes travel in the blood and anchor onto the vascular wall and migrate across into the organ or tissue side. Such altering of the membrane porosity can also be key in cancer research. Alternatively, the visual observation device can be any known device configured to facilitate visual observation of the biobarrier 12, such as a lens or a camera configured to capture images of the biobarrier 12. It will be appreciated and as described herein, that the window 114 passes through platform structure above the biobarrier 12.

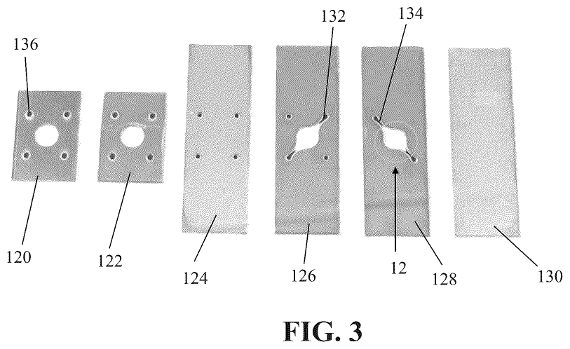

FIG. 3 illustrates an exploded top view of components of the HμB platform 100 having at least one frame 120, 122. In an example embodiment, a first frame 120 is to be positioned over a second frame 122, which in turn is positioned over a top electrode 124. A top channel member 126 is positioned below the top electrode 124. A bottom channel member 128 is positioned below the top channel member 126 with the biobarrier 12 disposed therebetween. A bottom electrode 130 is positioned below the bottom channel member 128. The one or more frames 120, 122 provide rigidity to the HμB platform 100. As shown, each of the frames 120, 122 has an opening that defines in part the window 114. These openings are aligned with and located above the biobarrier 12. As described herein, these components can be thought of as being layers of the overall HμB platform 100 which has a stacked arrangement.

The channels members 126, 128 can implement microchannels 132, 134, respectively, allowing fluids to pass through and be directed along a defined flow path. The microchannel 132 passes fluid above and about the biobarrier 12, and the microchannel 134 passes fluid below and about the biobarrier 12. In one embodiment, the top microchannel 132 acts as a capillary fluid flow path and the bottom microchannel 134 acts as an interstitial fluid flow path and, as described herein, the HμB platform 100 is designed so that the flowrates of the fluid in the top microchannel 132 and the fluid in the bottom microchannel 134 can be independently controlled. This permits, in the above example, the capillary fluid flowing in the top microchannel 132 to be at a higher flow rate than the interstitial fluid flowing in the bottom microchannel 134. For example, in the context of examining a biobarrier 12 modeling a retina as an iBRB, one pump, as described below, can provide a first fluid to pass through the top microchannel 132 at a flow rate of 3 μL/min., while another pump can provide a second fluid to pass through the bottom microchannel 134 at a flow rate of 1 μL/min. Alternatively, the flow rate through the top microchannel 132 can be less than the flow rate though the bottom microchannel 134.

In another example, the top microchannel 132 passes the first fluid, such as blood, to pass over and about the lumen 14. Alternatively, the first fluid can be composed of the combination of blood and a drug to be tested on the biobarrier 12. The bottom microchannel 132 passes the second fluid over and about the interstitial space 16. The second fluid can be water. Alternatively, the second fluid can be an aqueous solution.

Each of the components 120, 122, 124, 126 can include apertures, such as example aperture 136, to couple the inlets 106, 108 and the outlets 110, 112, respectively, shown in FIG. 2, to the microchannels 132, 134, respectively, allowing fluids to pass through. The top and bottom electrodes 124, 130 are connected to the conductive leads shown in FIG. 2. More particularly, as shown in 1, each of the frames 120, 122 include four apertures that surround a larger center hole that defines the window 114. Two of these apertures define first and second inlet flow paths and two define first and second outlet flow paths. It will also be appreciated that additional optional apertures can be provided for providing light to the biobarrier 12 to enhance imaging thereof. The top electrode 124 includes four apertures aligned with the apertures in frames 120, 122 and these four apertures define the first and second inlet flow paths and the first and second outlet flow paths. The channel member 126 can be thought of as being a top channel member and includes four apertures two of which define the inlet and outlet associated with the top microchannel 132, while the other two apertures are pass through openings that lead to the inlet and outlet associated with the bottom microchannel 134. The channel member 128 can be thought of as being a bottom channel member and includes only two apertures that define the inlet and outlet of the bottom microchannel 134. The bottom electrode 130 has no apertures.

Each of the components 120, 122, 124, 126 can be composed of a rigid material. Alternatively, each of the components 120, 122, 124, 126 can be composed of a semi-rigid material. For example, each of the components 120, 122, 124, 126 can be composed of plastic. By being composed of plastic components, the HμB platform 100 can be fabricated relatively inexpensively. Alternatively, each of the components 120, 122, 124, 126 can be composed of glass. Each of the components 120, 122, 124, 126, 128 can include apertures configured to receive fasteners to detachably secure the components 120, 122, 124, 126, 128, 130 to each other to form the HμB platform 100. The fabrication of such components 120, 122, 124, 126, 128, 130 can be accomplished using any known fabrication method. For example, at least the components 120, 122, 124, 126 can be laser printed or laser cut from acrylic sheets without the need for cleanroom facilities. Alternatively, at least the components 120, 122, 124, 126 can be fabricated using additive manufacturing such as three-dimensional (3D) printing.

In one embodiment, each of the components 120, 122, 124, 126, 128, 130 can be transparent plastic layers to allow imaging through the components 120, 122, 124, 126, 128, 130. By being composed of transparent plastic layers, the HμB platform 100 having the components 120, 122, 124, 126, 128, 130 allow for brightfield imaging, fluorescence imaging, or other known imaging methods, and the components 120, 122, 124, 126, 128, 130 can be manufactured using a laser cutter. In another embodiment, each of the electrodes 124, 130 can be composed of conductive plastic formed by bonding an acrylic sheet and a film. The acrylic sheet and the film can be transparent. The film can be composed of indium-tin-oxide. Alternatively, the film can be composed of any known film material.

In addition, the components 120, 122, 124, 126, 128, 130 can be detachably mounted to each other to form the HμB platform 100 using any known fastening devices, such as a vise, or can be clamped together with a clamp. By having detachably mounted components, the HμB platform 100 can be easily assembled, with the biobarrier 12 mounted therein. In another alternative embodiment, some or all of the components 120, 122, 124, 126, 128, 130 can be permanently mounted to each other to form the (stacked) HμB platform 100, for example, using fasteners, adhesive, glue, epoxy, and other known securing mechanisms or materials. The fasteners can be screws. The fasteners can be composed of materials which are not electrically conductive, in order to prevent the electrodes 124, 130 from being electrically connected and electrically shorted. For example, the fasteners can be composed of plastic. In a further alternative embodiment, the components 120, 122, 124, 126 can be permanently mounted to each other to form a first sub-assembly, and the components 128, 130 can be permanently mounted to each other to form a second sub-assembly. The biobarrier 12 can be positioned between the first and second sub-assemblies, and the first and second sub-assemblies can then be detachably secured to each other by any known detachable securing devices or materials, such as fasteners.

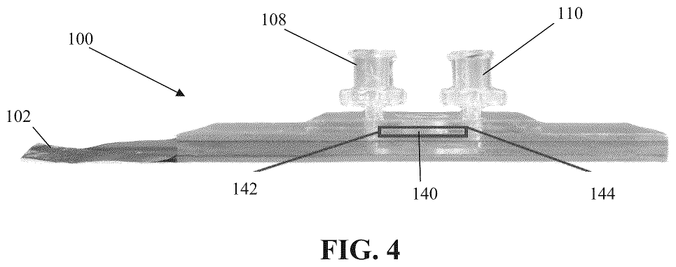

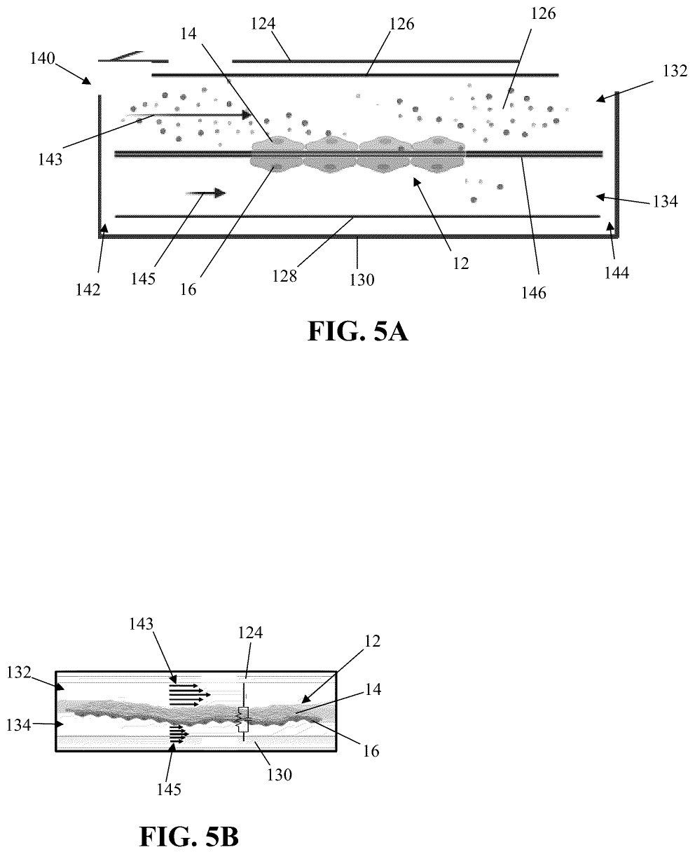

FIG. 4 illustrates a side elevational view of the HμB platform 100 of FIG. 2, with the components 120, 122, 124, 126, 128 shown in FIG. 3 stacked (layered) to form the HμB platform 100. As shown in FIGS. 3 and 4, as viewed from the side, an example inlet 108 and an example outlet 110 can be implemented using Luer-locks to provide a fluid passage from tubing (a conduit), as described below, to the apertures 136 and then to the microchannels 132, 134, as described above. The components 120, 122, 124, 126, 128 form the channel configuration 140 shown in greater detail in FIGS. 5A-5B, with an inlet portion 142 of the channel configuration 140 connected to the inlets 106, 108, and the outlet portion 144 of the channel configuration 140 connected to the outlets 110, 112. As shown in FIG. 3, the microchannels 132, 134 are oriented in an X shape with the biobarrier 12 being between one diagonal leg of the X and the other diagonal leg of the X. However, it will be understood that the orientation of the microchannels 132, 134 can be different in that they can be parallel to one another (in spaced apart parallel planes).

In addition, as shown, the top channel member 126 can be constructed such that the microchannel 132 has an enlarged center portion which corresponds to the location of the biobarrier 12 and is likewise aligned with the window 114. Similarly, the bottom channel member 128 can be constructed such that the microchannel 134 has an enlarged center portion which corresponds to the location of the biobarrier 12 and is likewise aligned with the window 114. The inlet and outlet of the respective microchannel can be formed on opposite sides of this enlarged center portion.

FIG. 5A illustrates a side cross-sectional view of a biobarrier 12 positioned in the HμB platform 100. As mentioned, the biobarrier 12 component of the HμB platform 100 has a lumen 14 (for carrying the first fluid) and an interstitial space 16 (for carrying the second fluid). The first and second fluids can be different or in some embodiments, they can be the same. As also discussed herein, the microchanneling of the HμB platform 100 permits fluid to flow at two different flow rates on opposite sides (faces) of the biobarrier 12. The biobarrier 12 can be positioned between the channels members (layers) 126, 128. In an alternative embodiment, the biobarrier 12 can be mounted on an intermediate member 146 positioned between the channel members 126, 128. The intermediate member 146 can be a porous membrane or other structure; however, in any event, the biobarrier 12 (membrane) is exposed along its top surface and its bottom surface.

The HμB platform 100 includes the top channel member 126 forming the top microchannel 132 configured to direct a fluid flow 143 over and about the lumen 14, and also includes the bottom channel member 128 forming the bottom microchannel 134 configured to direct a fluid flow 145 under and about the interstitial space 16. The first inlet 106 shown in FIG. 2 can be connected to the top microchannel 132 to direct the influx of fluid through the top microchannel 132, and the second inlet 108 shown in FIG. 2 can be connected to the bottom microchannel 134 to direct the influx of fluid through the bottom microchannel 134. Similarly, a first outlet shown in FIG. 2 can be connected to the top microchannel 132 to direct the outflow of fluid from the top microchannel 132, and the second outlet shown in FIG. 2 can be connected to the bottom microchannel 134 to direct the outflow of fluid from the bottom microchannel 134.

The fluid flow rates of the fluid flows 143, 145 through the top microchannel 132 and the bottom microchannel 134, respectively, can be tuned and controlled to be identical. Alternatively, the fluid flow rates of the fluid flows 143, 145 through the top microchannel 132 and the bottom microchannel 134 can be tuned and controlled to be different. In other words, the two flow rates are independently “tunable” and selectable. For example, as shown in FIG. 12, a fluid delivery device (e.g., a peristaltic pump or syringe pump) can be coupled to a media providing reservoir, with a controller capable of controlling the peristaltic pump to establish the fluid flow rates through the top microchannel 132 and the bottom microchannel 134 to be identical or to differ, to replicate physiological flow of fluids on, above, below, and about a biobarrier 12. Different flow rates can be used to model a diseased tissue or a physiological condition, such as high blood pressure or glaucoma. In the example of high blood pressure, such as in a BBB, the first fluid through the top microchannel 132 can have a higher flow rate than the second fluid through the bottom microchannel 134. Other physiological conditions in the BBB, in a gut barrier, a lung barrier, or other biobarriers or membranes can be modeled. Such different flow rates can be implemented using a peristaltic pump connected to the top microchannel 132 and a syringe pump connected to the bottom microchannel 134. Alternatively, the different flow rates can be implemented using a syringe pump connected to the top microchannel 132 and a peristaltic pump connected to the bottom microchannel 134. In another alternative embodiment, each microchannel 132, 134 can be connected to a respective pump of any known type. Alternative pumps can be compatible with a peristaltic pump or a syringe pump. In further alternative embodiments, both microchannels 132, 134 can be connected to separate peristaltic pumps, to separate syringe pumps, or to any other known pumps.

In the event that the first and second fluids are not the same, then two fluid delivery devices can be used to deliver the first fluid to the top microchannel 132 and the second fluid to the bottom microchannel 134. Even if the same fluid is used for both microchannels 132, 134, the two fluid delivery devices can be operated differently resulting in the different fluid flow rates through the two microchannels 132, 134.

FIG. 5B illustrates an alternative side cross-sectional view of the biobarrier positioned in the HμB platform 100, with the fluid flow 143 having a capillary blood flow rate Q₁ and the fluid flow 145 have an interstitial flow rate Q₂. For example, the capillary blood flow rate Q₁ is 3 μL/min., and the interstitial flow rate Q₂ is 1 μL/min. The electrodes 124, 130 have an associated barrier impedance |Z|. The HμB platform 100 is configured to replicate in vitro the cellular interface of an in vivo setting, with the ability to induce and control the physiological flow rates Q₁, Q₂ of the fluid flows 143, 145, respectively.

The outlets 110, 120 can also be used for sampling solutes and other elements and molecules that cross the biobarrier 12. The permeability of the biobarrier 12 can be determined by changes in the fluid flow, changes in the concentration of the sampled solutes, and other known methods of determining a permeability of a material such as the biobarrier 12. For example, the pressure experienced by the peristaltic pump or the rate of flow of the fluid in the media collecting reservoir can be measured by any known method to determine the permeability of a material such as the biobarrier 12. Alternatively, the concentration of the sampled solutes in the outflux of the fluid can be measured by any known concentration measuring device using a known method to determine the permeability of a material such as the biobarrier 12. The outlets 110, 120 can lead to a collection reservoir.

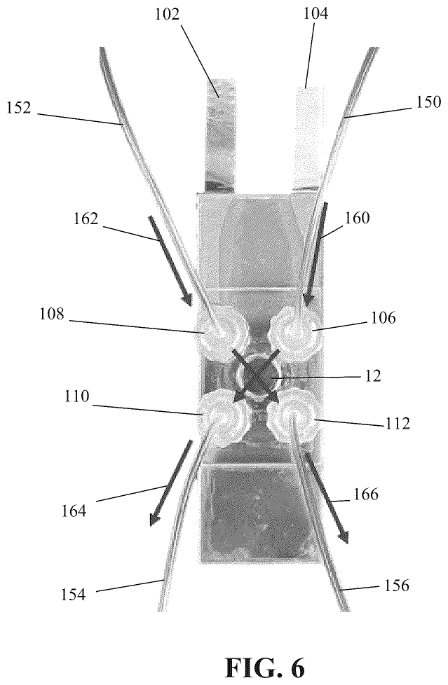

FIG. 6 illustrates a top plan view of the HμB platform 100 with the inlets and the outlets showing the flow of fluids through the HμB platform 100, with the tubing 150, 152, 154, 156 connected to the inlets 106, 108 and the outlets 110, 112, respectively, and with the biobarrier 12 positioned in the HμB platform 100. In one embodiment, the flow 160 of the fluid from the first inlet 106 through the top microchannel 132 and out the first outlet 110 as the flow 164 can be in a first direction at an angle to the flow 162 of the fluid from the second inlet 108 through the bottom microchannel 134 and out the second outlet as the flow 166, as shown in FIG. 6. Alternatively, the flows 160, 164 can be directed from the first inlet 106 through the top microchannel 132 and out the second outlet 112 as the flow 164, and the flows 162, 166 can be directed from the second inlet 108 through the bottom microchannel 134 and out the first outlet 110 as the flow 166, with the flows 160, 164 parallel to the flows 162, 166. Directional arrows in FIG. 6 depict the flow directions and patterns.

It will also be appreciated that the conduit connected to one or more of the inlets can be in the form of a wye tube which has a main leg for delivery of a base fluid and has branch leg for delivery of a drug or the like. In this example, the drug is delivered to the culture on biobarrier 12 and this permits monitoring cellular behavior to the drug. In addition, fluorescent molecules can be introduced to one of the microchannels to observe and monitor the permeability and integrity of the biobarrier 12 as by observing what amount of the fluorescent molecules are transferred across the biobarrier 12. In one embodiment, the HμB platform 100 is used to model the capillary wall of the retina. In one embodiment, in retina modeling, the flow rate in the top microchannel 132 can be 3 μL/min and the flow rate in the bottom microchannel 134 can be 1 μL/min. The fluid in the top microchannel 132 can be blood (optionally with a drug) and the fluid in the bottom microchannel 134 can be an aqueous solution.

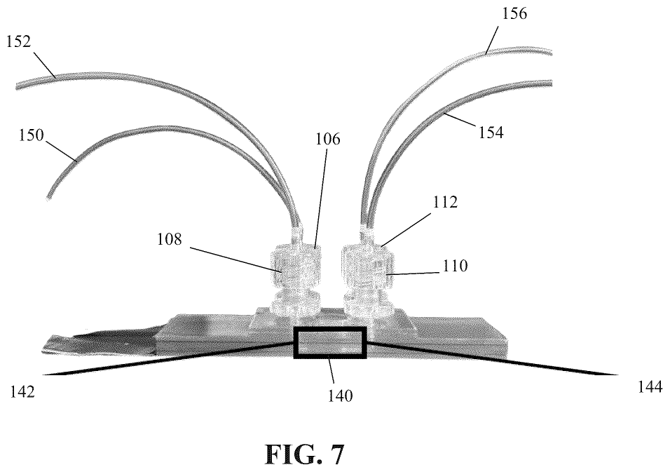

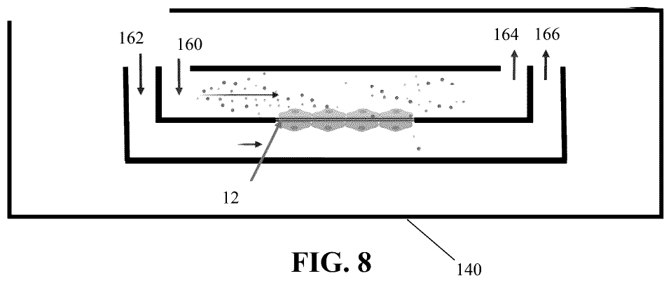

FIG. 7 illustrates a side elevational view of the HμB platform 100 of FIG. 6 with the tubing 150, 152, 154, 156 coupled to respective inlets 106, 108 and outlets 110, 112. FIG. 8 illustrates a side cross-sectional view of the biobarrier 12 with the flows 160, 162, 164, 166 of fluids through the tubing 150, 152, 154, 156, the inlets 106, 108, and outlets 110, 112, respectively, and the channel configuration 140.

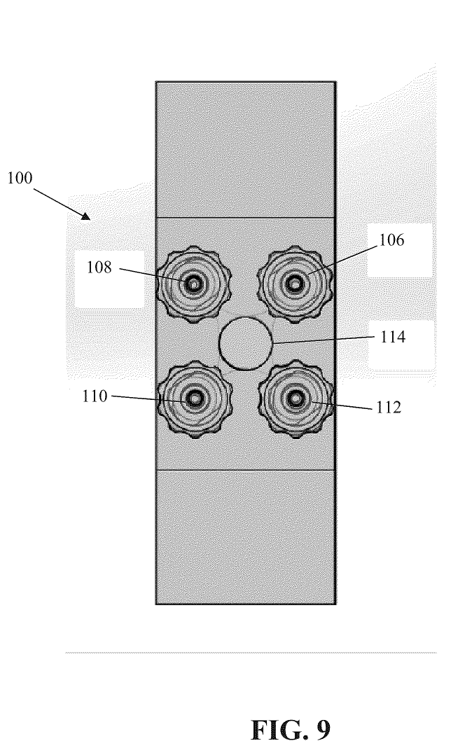

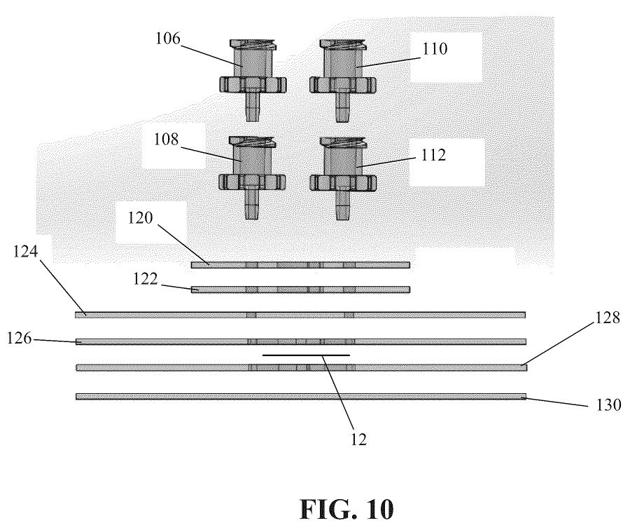

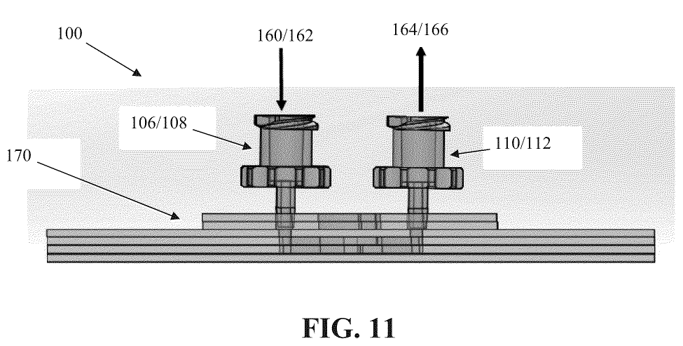

FIG. 9 illustrates a top plan view of the HμB platform 100 of FIG. 2 without the conductive leads 102, 104 in FIG. 2. FIG. 10 illustrates a side exploded view of the HμB platform 100 of FIG. 9, showing the various components 120, 122, 124, 126, 128, 130 of FIG. 3 stacked with the biobarrier 12 disposed between the channel members 126, 128. FIG. 11 illustrates a side cross-sectional view of the HμB platform 100 of FIG. 9 with the inlets 106, 108, the outlets 110, 112, and the various components 120, 122, 124, 126, 128, 130 in an assembled configuration 170, having the inflow 160, 162 of liquid, and the outflow 164, 166 of liquid passing through the HμB platform 100.

FIG. 12 illustrates a top front view of a system 200 having the HμB platform 100 at the incubator stage between the inlets 106, 108 and the outlets 110, 112. A microscope can be disposed over the HμB platform 100 with the microscope configured to observe the biobarrier 12 positioned in the HμB platform 100 at the incubator stage. The microscope can be an upright microscope. Alternatively, the microscope can be an inverted microscope. The components 120, 122, 124, 126, 128, 130 can be composed of transparent or translucent materials, which facilitate brightfield and fluorescence microscopy. A plurality of peristaltic pumps can be coupled to media providing reservoirs and also coupled to the channel members 126, 128 to provide the flow of fluid to respective inlets 106, 108. A plurality of collecting reservoirs are coupled to the outlets 110, 112 and configured to receive the flow of fluid from the channel members 126, 128 through the respective outlets 110, 112. An electrically conductive connection can electrically couple the conductive leads 102, 104 shown in FIG. 2 to an electrical analyzer, such as an impedance analyzer.

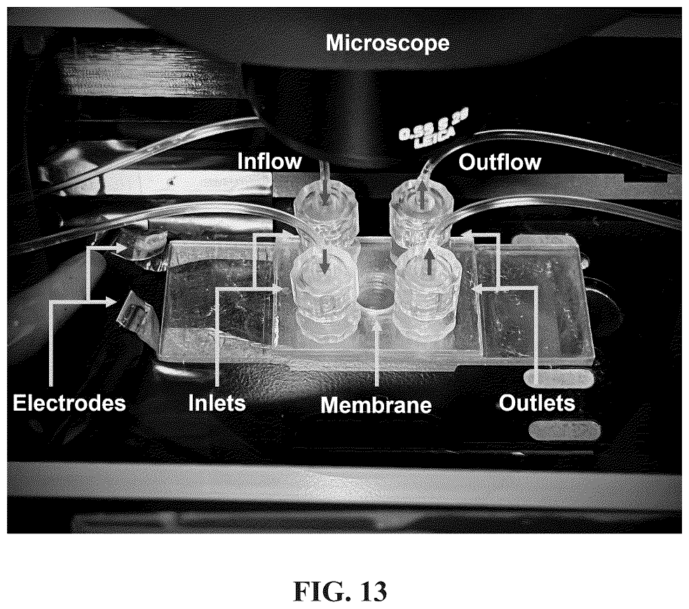

FIG. 13 illustrates a top perspective view of the HμB platform 100 in the system 200 of FIG. 12. The biobarrier 12 can be a porous membrane having a porosity of, for example, 0.4 μm. The inlets 106, 108 and the outlets 110, 112 of the HμB platform 100 are configured to provide the flow of fluid over, under, across, and about the biobarrier 12. For example, cells can be placed on a porous intermediate membrane 146, as shown in FIGS. 5A-5B, to form the biobarrier 12 having a lumen 14 and an interstitial space 16. The cells can be provided in the fluids passing through either or both of the microchannels 132, 134 after injection in the inlets 106, 108. The porous intermediate membrane 146 can serve as a scaffold for cell seeding on each side of the porous intermediate membrane 146, effectively forming tissue barriers and biobarriers 12.

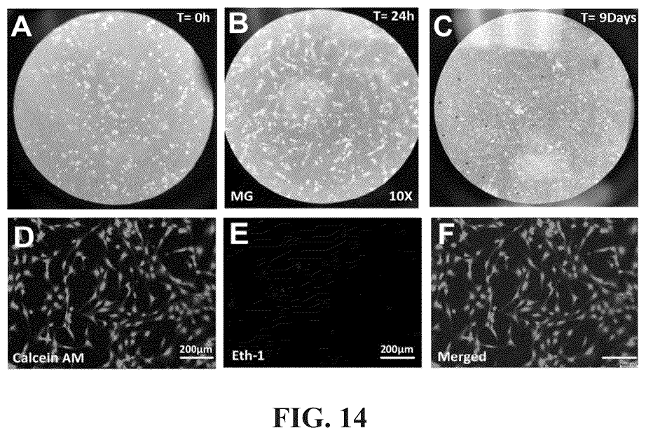

FIGS. 14A-14F illustrate various views of cultures of Muller glia on a biobarrier 12 observed using the HμB platform 100. FIG. 14A shows Muller glia on the porous biobarrier 12 at time t=0 hours. FIG. 14B shows the Muller glia under static conditions after t=24 hours. FIG. 14C shows the Muller glia under flow conditions after t=48 hours. FIG. 14D shows live cells using a Calcein AM fluorescent dye. FIG. 14E shows dead cells using an ethidium HD-1m as a fluorescent tag. FIG. 14F shows a merge of the images of FIGS. 14D and 14E.

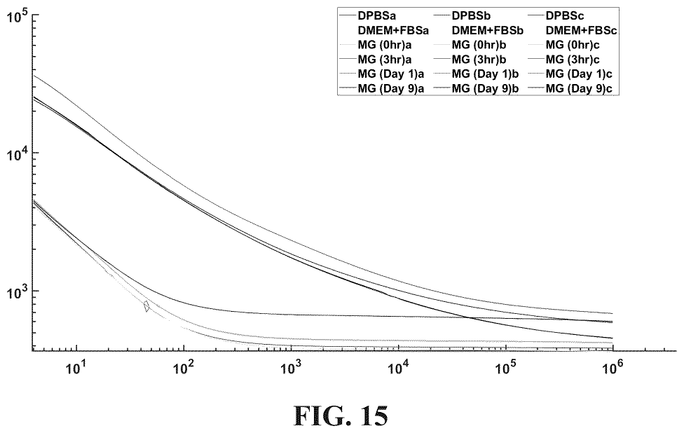

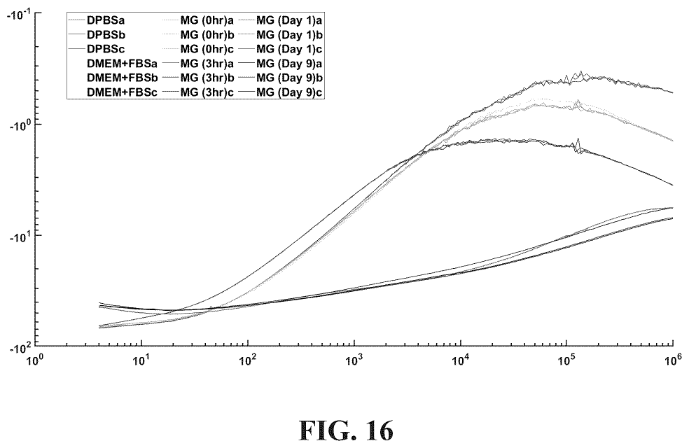

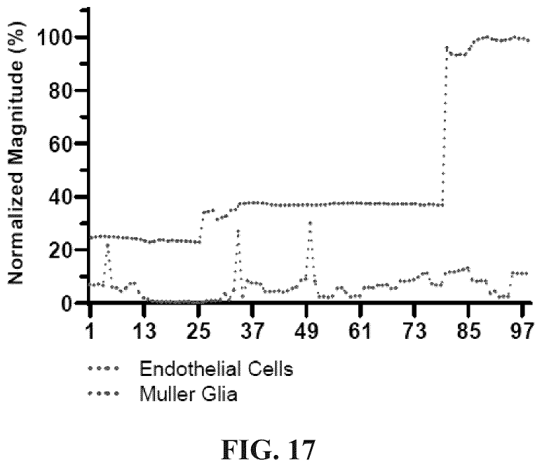

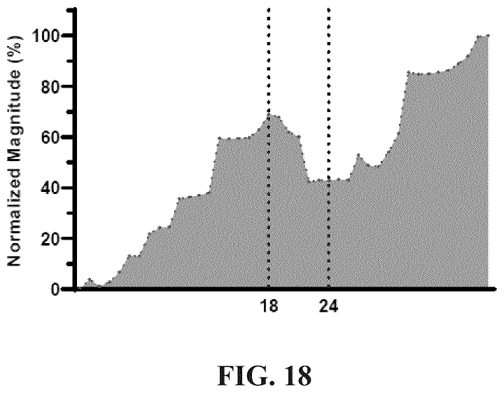

FIG. 15 illustrates a graph of a magnitude of a current opposed by the resistance and capacitance of Muller glia monolayers, determined using the impedance analyzer of the system 200 receiving the currents from the HμB platform 100 through the conductive leads 102, 104 shown in FIGS. 2 and 12. FIG. 16 illustrates a graph of a phase diagram of Muller glia monolayer impedance, determined using the impedance analyzer of the system 200 receiving the currents from the HμB platform 100 through the conductive leads 102, 104 shown in FIGS. 2 and 12. FIG. 17 illustrates a graph of normalized impedance of Muller glia and endothelial cells, determined using the impedance analyzer of the system 200 receiving the currents from the HμB platform 100 through the conductive leads 102, 104 shown in FIGS. 2 and 12. FIG. 18 illustrates a graph of normalized impedance of endothelial cells, determined using the impedance analyzer of the system 200 receiving the currents from the HμB platform 100 through the conductive leads 102, 104 shown in FIGS. 2 and 12.

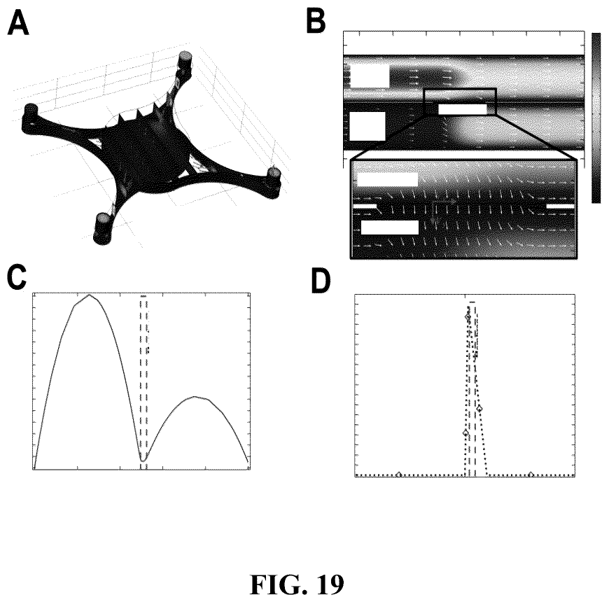

FIG. 19A illustrates a three-dimensional computer simulation of the fluid flow over, under, and around the biobarrier 12 positioned in the HμB platform 100. FIG. 19B illustrates a computer model of fluid velocity in the microchannels 132, 134 of the HμB platform 100, as shown in FIG. 3, and across the biobarrier 12. FIG. 19C illustrates a computer model of a velocity profile across each of the microchannels 132, 134. FIG. 19D illustrates a computer model of a shear stress profile across the biobarrier 12.

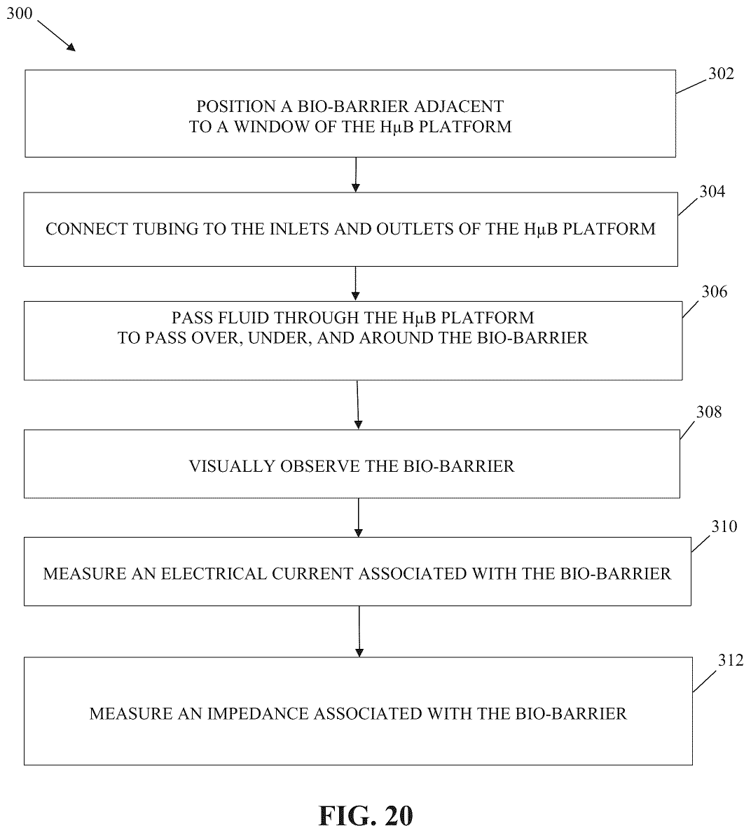

As shown in FIG. 20, a method 300 of operation of the HμB platform 100 and the system 200 includes the step of positioning a biobarrier 12 adjacent to a window 114 of the HμB platform 100 in step 302, connecting the tubing 150, 152, 154, 156 to the inlets 106, 108 and the outlets 110, 112, respectively in step 304, and pass fluid through the HμB platform 100 to pass over, under, and around the biobarrier 12 in step 306. The method 300 can further include visually observing the biobarrier 12 in step 308, for example, using the microscope in FIG. 12. The method 300 can further include measuring an electrical current associated with the biobarrier 12, for example, through the conductive leads 102, 104 using the impedance analyzer in FIG. 12 in step 310. The method 300 can further include measuring an impedance or voltage associated with the biobarrier 12, for example, through the conductive leads 102, 104 using the impedance analyzer in FIG. 12 in step 312. Accordingly, characteristics and properties of the biobarrier 12 including visual appearances, currents, voltages, and impedances of or across the biobarrier 12 can be measured and evaluated.

### Additional Embodiments

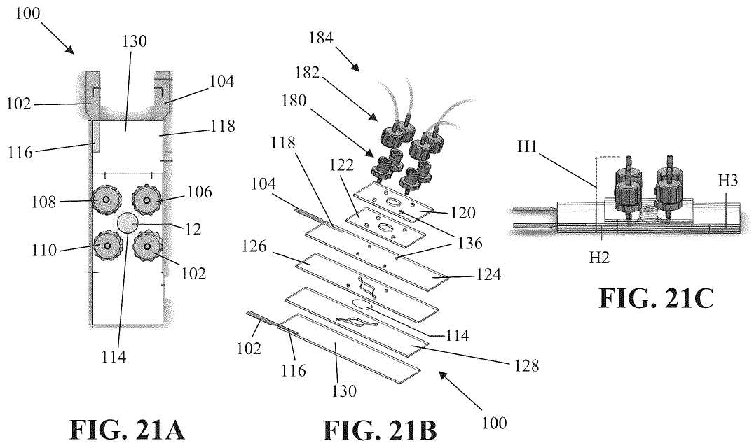

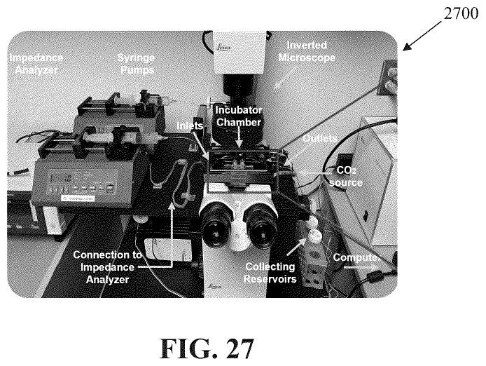

The HμB platform 100, the system 200, and the method 300 described above permit an in vitro examination of the biobarrier 12. Further aspects of the HμB platform 100, the system 200, and the method 300 are described below with reference to FIGS. 21A-36. As shown in FIGS. 21A-21C, the HμB platform 100 has a semi-transparent multi-layered geometry featuring two independent inlets 106, 108 and outlets 110, 112 separated by a porous membrane 12. Pressure drives fluid flow through the inlets 106, 108 into two channels 132, 134 separated by a porous membrane 12, as shown in FIGS. 5A-5B. Fluid travels along and through the porous membrane 12 and exits at the outlets 110, 112 into collecting containers or reservoirs of the system 200, as shown in FIG. 12, or the system 2700 shown in the alternative embodiment in FIG. 27. Such systems 200, 2700 include two conductive clear electrodes 124, 130 that record the impedance (resistance and capacitance) across the porous membrane 12. The HμB platform 100 allows cell-seeding on both sides of the membrane 12, which can remain in static conditions while cells are growing. Once cells have attached, flow can be introduced at physiological flow rate conditions to supply nutrients to the cells, while the electrodes 124, 130 can record cell-based impedance changes over time. Additionally, the semi-transparent layers of the HμB platform 100 permit brightfield and fluorescence imaging via an inverted microscope.

The HμB platform 100 was designed using computer-aided drafting (or design) (CAD), for example, in SolidWorks, featuring dimensions of a standard microscope slide, namely 76 mm (7.6 cm.) long×26 mm (2.6 cm.) wide, as shown in FIG. 2. As shown in FIGS. 21A-21C, the HμB platform 100 includes the layers of components such as the top electrode 124, the top channel layer 126, the bottom channel layer 128, and the bottom electrode 130, with the channel layers 126, 128 separated by a porous membrane 12, for example, a polyester track etch (PETE) membrane, for example, 12 μm thick with pores, for example, 0.4p m in diameter. The top electrode 124 and the top channel layer 126 are bonded or attached to at least one frame 120, 122 that stabilize the collection 180 of inlets 106, 108, and outlets 110, 112, which then each connect to respective Luer-lock fittings 182 attached to the respective tubings 184 through where fluid flows, as shown in FIG. 21B. In one embodiment, the components 120, 122, 124, 126 include apertures to respectively receive fasteners to assembly the components 120, 122, 124, 126 together as the HμB platform 100. In one embodiment, the fastener is a screw engaging a respective aperture. Since at least the layers 124, 130 are electrically conductive, the fasteners such as a screw is composed of a non-conductive material, such as plastic. In an alternative embodiment, the components 120, 122, 124, 126 are assembled together and held together using a vise or other known fastening mechanisms, as described above.

In one embodiment, all the main layers 124, 126, 128, 130 and frames 120, 122 of the HμB platform 100 are composed of transparent acrylic with a thickness, for example, 1 mm each. As shown in FIGS. 21A-21B, the leads 102, 104 extend from the assembly of layers 124, 126, 128, 130, with the bases 116, 118 of the leads 102, 104 attached to the electrodes 130, 124, respectively. The channel layers 126, 128 have a mirrored geometry featuring one inlet and one outlet of 1 mm in diameter connected by a 7.8 mm diameter chamber. When the channel layers 126, 128 are placed one on top of the other, the geometry creates an “X” shape, only divided by the porous membrane 12. The X-geometry enables two independent inlets 106, 108 and outlets 110, 112 with separate fluid flows 143, 145 to interface at the porous membrane 12, where fluid flows along the membrane 12 and percolates through the pores and exits via the outlets. The porous membrane 12 serves as a hub for cell growth on either side of the membrane 12 in the presence of constant flow, as depicted in FIGS. 5A-5B.

In an example embodiment, the electrodes 124, 130 are composed of acrylic sheets bonded on one side to a film of polyethylene terephthalate (PET) coated with a layer of indium tin oxide (ITO) of 0.175 mm thick. As shown in FIGS. 21A-21B, in one embodiment, the upper left corner on the conductive side (the ITO side) of the electrode 130 is bonded to a copper foil electrical tape as the lead 102 with the base 116 to provide an electrical connection to the outside of the HμB platform 100 once the HμB platform 100 is assembled from the components 120, 122, 124, 126, 128, 130. The upper right corner on the conductive side (the ITO side) of the electrode 124 is bonded to a copper foil electrical tape as the lead 104 with the base 118 to provide an electrical connection to the outside of the HμB platform 100.

The top electrode 124 features, for example, four equidistant holes of 1 mm diameter that match the inlets 106, 108 and outlets 110, 112 at the positions in the top channel layer 126 corresponding to the inlets 106, 108 and outlets 110, 112. The electrode layers 124, 130 “sandwich” the layers 126, 128 of the HμB platform 100, creating a closed system only open to the atmosphere through the inlets 106, 108 and outlet 110, 112 corresponding to the apertures 136 in the top electrode layer 124. The leads 102, 104 can then be connected to an impedance analyzer to evaluate the impedance across the porous membrane 12 in the presence of a liquid medium. Hence, the HμB platform 100 provides an efficient way to measure the impedance of cell barriers on the porous membrane 12 in the presence of fluid flow over time, while enabling brightfield and fluorescence imaging through the clear electrode layers 124, 130.

The HμB platform 100 is designed to be compatible with a dual microscope slide insert, such as the product associated with the product code 2×GS-M obtained from Okolab, that fits inside of an incubator chamber such as the product associated with the product code H201-K-FRAME obtained from Okolab, sitting on top of the stage of an inverted microscope such as the product associated with the product code DMi8 obtained from Leica Technologies. As shown in FIG. 21C, in an example embodiment, the overall height H1 of the HμB platform 100, including the Luer locks attached to the inlets 106, 108 and the outlets 110, 112, is 3 cm. In the example embodiment, the overall height H2 of the stack of all of the layers 120, 122, 124, 126, 128, 130 is 0.60 cm., and the overall height H3 of the stack of the layers 124, 126, 128, 130 is 0.60 cm. As described below, an incubator maintains the HμB platform 100 at 37° C. and 5% CO₂.

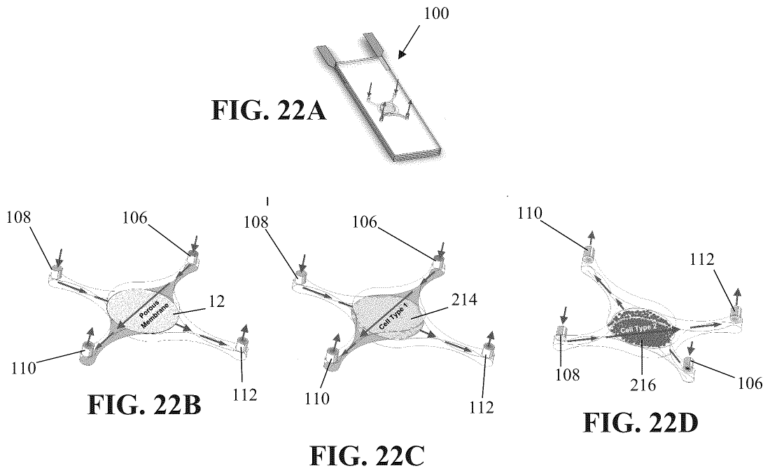

FIGS. 22A-22D illustrate fluid flows into and out the channels of the HμB platform 100, with red arrows showing the flow direction in the top channel 132, while blue arrows display the flow direction in the bottom channel 134, as shown in FIGS. 5A-5B. Additionally, FIGS. 22B-22D illustrates how two cell types can attach and form a cell barrier 214, 216 on either side of the porous membrane 12 while experiencing continuous flow.

Fluid flow in the HμB platform 100 is defined by a pressure driven flow in a free and porous medium. Specifically, the pressure is exerted by the syringe pumps driving the fluid freely through the channels 132, 134, while fluid also percolates through the porous membrane 12. Hence a combination of the Navier-Stokes equation, which describes free flow of an incompressible Newtonian fluid, and Darcy's law that describes the flow of fluid in porous media driven by a pressure gradient, is necessary to define the fluid dynamics in the HμB platform 100. The Navier-Stokes Equation (1), the Continuity Equation (2), and Darcy's law Equation (3) are defined below:

$$ρ ( ( \frac{\partial \overline{u}}{\partial t} ) + ( \overline{u} \cdot ∇ ) \overline{u} ) = - ∇ p + μ {∇}^{2} \overline{u} + \overline{F} \qquad ( 1 )$$

$$∇ \cdot \overline{u} = 0 \qquad ( 2 )$$

$$∇ p = - \frac{μ}{κ} \overline{u} \qquad ( 3 )$$

where ρ is the density of the fluid, t is time, ∇ is the gradient operator,

$$( \frac{\partial \overline{u}}{\partial t} ) + ( \overline{u} \cdot ∇ ) \overline{u}$$

is the change in velocity over time plus the speed and direction of the fluid, ∇p is the pressure gradient, μ∇²ū is the term for viscous forces, F̅ is the term for external forces such as gravity, and κ is the permeability constant of the porous medium (membrane). Applying the Navier-Stokes equations to a free and porous medium at steady state, neglecting external forces, and including Darcy's law, we obtain Brinkman's Equation (4):

$$0 = - ∇ p + \frac{μ}{{ε}_{p}} + \frac{μ}{κ} \overline{u} \qquad ( 4 )$$

where −∇p is the pressure gradient driving the motion of the fluid, εₚ is the porosity of the medium, and

$$\frac{μ}{κ} \overline{u}$$

is Darcy's law. Brinkman's Equation (4) is an extension of the Navier-Stokes equations with the addition of Darcy's law. This Equation (4) describes the motion of a pressure driven fluid moving in a bound free space, such as the channels 132, 134, while also moving through with a medium of high porosity, such as the membrane 12.

The mass transport of solutes traveling in the medium along and across the porous membrane 12 is governed by the Convection-Diffusion equation, which describes the movement of particles by a convective force (fluid velocity) and a diffusive force (Fick's Law) inside the HμB platform 100. The Convection-Diffusion Equation (5) is defined below:

$$\frac{\partial C}{\partial t} = \overline{u} \cdot ∇ C + {DV}^{2} C \qquad ( 5 )$$

where C is the concentration of the solute, t is time, ū·∇C is the convective term defined by the fluid velocity ū, ∇ is the gradient operator, D is the diffusion coefficient of the solute, and ∇² is the Laplacian. At steady state conditions the change in concentration with respect to time is zero (

$$\frac{\partial C}{\partial t} = 0 .$$

The velocity in the convective term is the same as in the Darcy's law in Equations (3) and (4). Further, the momentum of the solute particles in this equation are governed by the Brinkman's Equation (4).

The fluid mechanics in the HμB platform 100 were modeled in COMSOL Multiphysics 5.3a using the physics of “Free and Porous Media Flow (fp)”, governed by the Brinkman's Equation (4), described above. A three-dimensional (3D) model was developed to illustrate the fluid behavior inside the HμB platform 100 in steady state conditions, while a two-dimensional (2D) model was developed to characterize fluid velocity and shear stress within the channels 132, 134. The geometry of the HμB platform 100 was developed using computer aided drafting (or design) (CAD) in SolidWorks and supplemented with the built-in geometry library of COMSOL. Water was used as the test fluid for these simulations, since water has the same density and viscosity as a cell culture medium, which is the fluid intended to be used in the HμB platform 100. Polycarbonate was used as the material for the porous membrane 12 with its physical properties derived from the COMSOL built-in library. The fluid velocity for the channels 132, 134 was derived from the average micro capillary flow rate (3 μL/min) and average interstitial flow rate (1 μL/min) in the body, respectively. The porosity (ε) and permeability (κ) of the membrane 12 were derived using Darcy's law above from readily available values such as a pore diameter (0.4 μm), a pore area (0.13 μm²), a thickness of the membrane 12 (12 μm), an amount of open area (33%), a pressure change (10 PSI), and physical properties such as flow rate per area (33 mL/min/cm²). The study was carried out in steady state conditions (stationary), which are the intended conditions where studies of fluid flow will be carried out in the HμB platform 100. The magnitude velocity was computed across the channels 132, 134 and the porous membrane 12, and the shear stress was derived from the shear rate multiplied by the viscosity of the fluid. Arrow surface plots were also simulated to display the direction of the flow at respective flow velocities along and across the porous membrane 12.

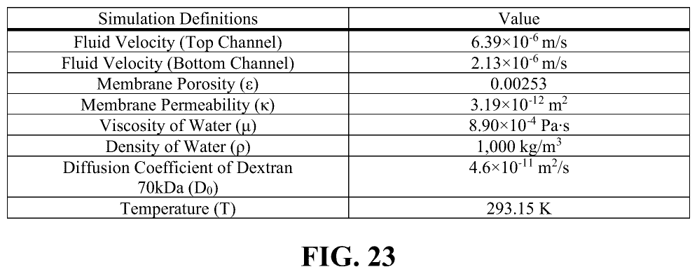

Mass transport in the HμB platform 100 was modeled in COMSOL Multiphysics 5.3a using two physics parameters: (1) the “Free and Porous Media Flow (fp)”, governed by the Brinkman's Equation (4), described above; and (2) the “Transport of Diluted Species (tds)”, governed by the Convection-Diffusion Equation (5), described above. The mass transport simulation was carried out in a 2D model to illustrate the movement of solutes along and across the porous membrane 12. The geometry of the HμB platform 100 was developed using the built-in geometry library of COMSOL. Water was used as the fluid in which the solutes travels, due to its similar viscosity and density properties as cell culture medium. The solute used for these simulations was dextran of 70 kDa with a diffusion coefficient (Do) of 4.6×10⁻¹¹ m²/s. The study was performed in a time-dependent condition to observe the changes of concentration across the porous membrane 12 over time. The simulation was run for five days with a time-steps of fifteen minutes, which is the intended time to measure the permeability of cells inside the HμB platform 100. The concentration over time was computed in the channels 132, 134 and across the membrane 12. A contour plot was graphed to clearly divide the concentration gradient in space, while an arrow surface plot was graphed to display the direction of the solute moving in space. Example physical properties and values used for these simulations are detailed in the table shown in FIG. 23.

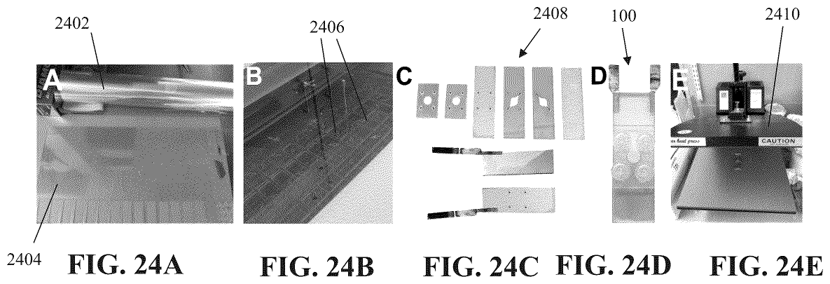

As shown in FIGS. 24A-24E, in an example embodiment, the HμB platform 100 is fabricated from acrylic sheets. As shown in FIG. 24A, a roll 2402 provides acrylic sheets 2404 provides of 60.96 cm. in length, 30.48 cm in width, and a 0.076 cm thickness, which can be the product associated with the product code ACRYCLR0.030IM24X48 obtained from ePlastics. The acrylic sheets 2404 are bonded to a double-sided tape on both sides, with the double-sided tape which can be the product associated with the product code 90727A140 obtained from McMaster. As shown in FIG. 24B, portions 2406 of the acrylic sheet in to be the components 2408 of the HμB platform 100, shown in FIG. 24C, are laser-cut from the acrylic sheets 2404 shown in FIG. 24A using a laser cutter, such as the product associated with the product code Zing 24 from Epilog. The components 2408 in FIG. 24C are then stacked to be assembled, as shown in FIG. 24D. The double-sided tape on the side where the channel layers 126, 128 meet was peeled off, and a 13 mm diameter porous polyester track etch (PETE) membrane 12, such as the product associated with the product code PET0413100e from Sterlitech, was placed on top of the 7.8 mm chamber on the sticky side of the bottom channel layer 128, following by the top channel layer 126 placed on top.

Then the channel layers 126, 128 were pressed together using a heat press 2410 as shown in FIG. 24E, at 120° C. for 1 minute to ensure bonding of the two layers. In one embodiment, the pressing step is repeated. The electrode layers 124, 130 were made using the same acrylic sheets 2404 of FIG. 24A, and using double-sided tape procedure for fabricating the channel layers 126, 128. However, for the electrode layers 124, 130, one of the sides of the acrylic sheet 2404 was bonded to an indium tin oxide (ITO) film, obtained from MSE Supplies, having a 70-100 Ω/sq. resistance. Then, the electrode layers 124, 130 were laser-cut as shown in FIG. 24B, and on the top left corner, a lead of copper foil electrical tape, such as the product associated with the product code 76555A641 obtained from McMaster, are bonded. The electrode layers 124, 130 were individually tested for conductance using a 32-volt DC power supply, such as the product associated with the product code PPS2320A from Circuit Specialists, and then bonded to the channel layers 126, 128 with the conductive side facing the channels 132, 134, and pressed together in the heat press 2410 in FIG. 24E. The frame layers 120, 122 were aligned with the apertures 138 of the inlets 106, 108 and the outlets 110, 112 on the top electrode layer 124, and pressed again using the heat press 2140.

Inlet inserts, such as the product associated with the product code 51525K281 obtained from McMaster, were press-fit into the inlets 106, 108 and the outlets 110, 112 of the HμB platform 100, followed by a thin spread of epoxy between the insert fittings 184 and the inlet/outlet apertures 136 to secure the fittings 184 and to prevent leakage. In one embodiment, a thin layer of epoxy was spread on the sides of the HμB platform 100, including the region where the frames 120, 122 meet the top electrode layer 124. The HμB platform 100 was left to air dry, followed by introducing ethanol 70% via the inlets 106, 108 using a 1 mL syringe such as the product associated with the product code 53548-001 obtained from VWR, to verify fluid flow. The HμB platform 100 is then exposed to ultraviolet (UV) light for one hour inside of a flow hood. Any ethanol 70% inside the HμB platform 100 was removed using a Pasteur pipet and the inlets 106, 108 and the outlets 110, 112 were capped using caps such as the product associated with the product code 51525K244 obtained from McMaster. The HμB platform 100 was stored inside of a 10 mm petri dish inside of a flow hood until needed.

Separately, silicon tubing, such as the product associated with the product code NC0578437 obtained from FisherScientific, having a 0.5 mm inner diameter was connected to a tube coupling cap such as the product associated with the product code 51525K271 obtained from McMaster, so that the Luer-lock side could couple to the inserts of the inlets 106, 108 and the outlet 110, 112 in the HμB platform 100. In one embodiment, a hand-drill with a diamond bit was used to enlarge the diameter of a spout of the coupling cap to press-fit the tubing, followed by a thin coat of epoxy at this interface to prevent leaking and to prevent inadvertent disassembling. For each HμB platform 100, a total of four sets of tubing 184 and coupling caps were manufactured to connect to the two inlets 106, 108 and the two outlets 110, 112 of the HμB platform 100. These sets of tubings 184 were sterilized and stored in a solution of ethanol 70% until use.

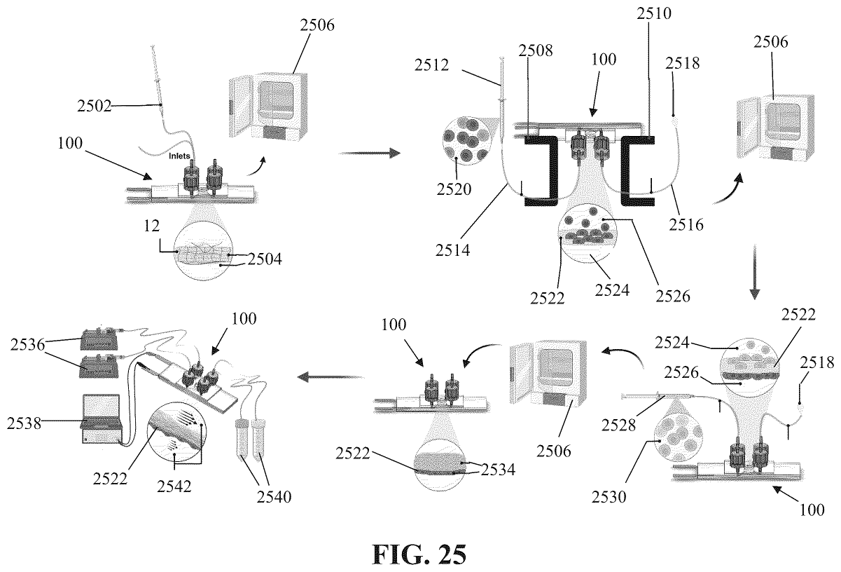

As shown in FIG. 25, a sequence of steps for seeding the cells in the HμB platform 100 and then operating the HμB platform 100 is illustrated. After the HμB platform 100 was sterilized with ethanol 70% and exposed to UV light under a biosafety cabinet such as a flow hood for one hour prior to any experiment. The HμB platform 100 was flushed in sterile conditions with Dulbecco's phosphate-buffered saline (DPBS), such as the product associated with the product code D8537 obtained from Sigma-Aldrich, and then aspirated using a Pasteur pipet. With the membrane 12 mounted in the HμB platform 100, a solution of Collagen IV, such as the product associated with the product code C6745 obtained from Millipore Sigma, and fibronectin, such as the product associated with the product code F0895 obtained from Millipore Sigma, which is diluted to a concentration of 10 μg/mL in DPBS was introduced to both channels 132, 134, using the syringe 2504, so that the porous membrane 12 mounted in the HμB platform 100 would be coated on both sides 2504, as shown in FIG. 25. The HμB platform 100 having the coating of the membrane 12 was placed inside of a petri dish and incubated at 37 C and 5% CO₂ overnight in an incubator 2506.

The matrix solution in the HμB platform 100 was aspirated using a Pasteur pipet, and the HμB platform 100 was flushed with DPBS. To prevent fluid flow, the tubing 2514, 2516 leading to the inlets 106, 108 and outlets 110, 112, respectively, of the HμB platform 100 were clamped close to the inserts, for example, using four metallic spring clamps of 4 cm in length, with the clamps such as the product associated with the product code RI-Shimeyao-49 obtained from Shimeyao. Dulbecco's modified Eagle's medium (DMEM) was then introduced into the HμB platform 100 through the inlets 106, 108, avoiding the formation of bubbles. The HμB platform 100 and the inlets 106, 108 were flushed with DPBS. The tubing 2414 was attached to the inlets 106, 108 of the HμB platform 100, and the tubing 2416 was attached to the outlets 110, 112 of the HμB platform 100. The tubing 2516 has an open end 2518.

The HμB platform 100 was flipped upside down, and the edges of HμB platform 100 were placed on a flat platform having surfaces 2508, 2510, for example, about 10 cm above the surface of a flow hood, while the tubing 2514, 2516 hung freely. In one embodiment, the edges of the HμB platform 100 were secured in place using a fastener, for example, tape. A syringe 2512 containing a solution 2520 of Muller glia (MG) in complete media at a concentration of 1.0×110⁶ cells/mL was introduced into the tubing 2514 leading to the bottom inlet until the solution came out through the bottom outlet, which guarantees that MG will attach to the bottom side of the membrane 2522.

The quantity 2520 includes only Muller glia (MG) which are introduced into the HμB platform 100 by the syringe 2512 to seed one side of the membrane 2522 in the HμB platform 100 for experiments that only require MG, with the coated membrane 2522 positioned between the top channel 2524 and the bottom channel 2526. In another embodiment, the quantity 2520 includes only endothelial cells (ECs) which are introduced into the HμB platform 100 by the syringe 2512 to seed one side of the membrane 2522 in the HμB platform 100 for experiments that only require ECs. The bottom inlets 106, 108 and outlets 110, 112 were clipped, and the HμB platform with the seeded membrane 2522 was placed inside of a mammalian incubator 2506 at 37° C. and 5% CO² for three hours.

After three hours, the HμB platform 100 was removed from the incubator 2506, and was taken inside of a flow hood, and flipped upright with the inlets 106, 108 and the outlets 110, 112 facing up. The HμB platform 100 was placed in a petri dish. Cell attachment to the membrane 2522 was verified using an inverted microscope via brightfield imaging. Inside the flow hood, the tubings 2514, 2516 connected to the top inlets 106, 108 and outlets 110, 112 were unclipped.

For combinatory experiments using both MG and ECs, the MG and ECs are both introduced by attaching a syringe 2528 to the tubing 2514, and the tubing 2516 has an open end 2518. Using the attached syringe 2528, a solution 2530 of ECs in complete media at 1.0×10⁶ cells/mL was introduced via the top inlet until the solution came out the top outlet. The tubings 2514, 2516 were then disconnected from all the inlets 106, 108, and all of the outlets 110, 112, and the HμB platform 100 was placed in a petri dish to check for successful cell seeding on the membrane 2522. The HμB platform 100 inside of the capped petri dish with the open inlets 106, 108 and the open outlets 110, 112 was placed in the incubator 2506 until full monolayers 2534 were formed on both sides of the membrane 2522. The monolayers 2534 were checked under the microscope every day, and media was replenished dropwise at the inlets using a 1 mL syringe.

Once full monolayer 2534 is formed on either one or both sides of the membrane 2522, the HμB platform 100 is placed in an incubator chamber 2506 for final incubation. After final incubation, the HμB platform 100 with the membrane 2522 seeded with full monolayers of cells on one or both sides of the membrane 2522, the HμB platform 100 is ready for experiments. The inlets 106, 108 are connected to syringe pumps 2536, and the leads 102, 104 of the HμB platform 100 are electrically connected to an impedance analyzer 2538. The tubing connected to the outlets 110, 112 will lead to conical tubes 2540 that work as collecting reservoirs. The syringe pumps 2536 apply the fluids to the inlets 106, 108, causing the flows 2542 of fluids to pass the seeded membrane 2522, while the impedance analyzer 2538 measures impedance, such as resistance and capacitance, across the seeded membrane 2522.

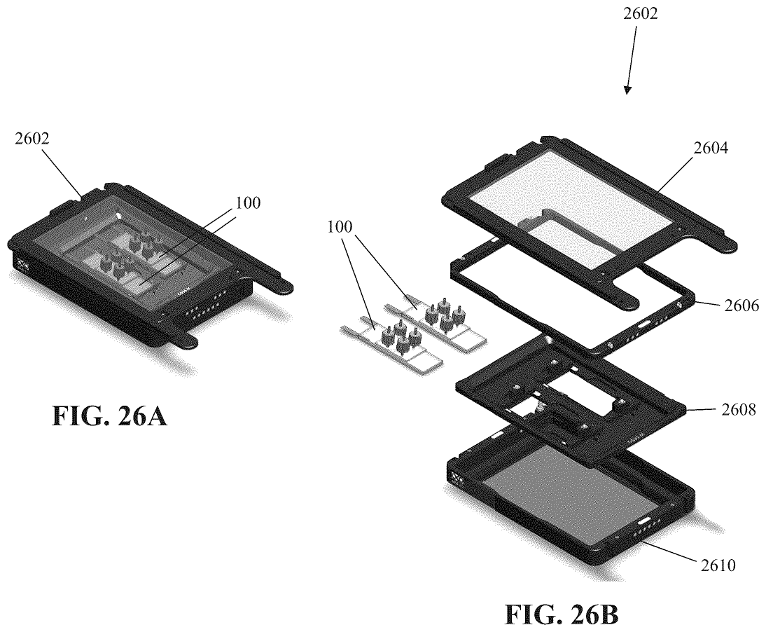

As shown in FIGS. 26A-26B, the HμB platform 100 is sized, configured, and dimensioned to fit within an incubation device 2602. In one embodiment, the incubator device 2602 with at least one HμB platform 100 inside is placed into the incubator 2506, with the incubator 2506 having heating coils and other components to heat and otherwise prepare the membrane 12, 2522 mounted in the HμB platform 100 for experimentation after incubation. In another embodiment, the incubator device 2602 includes such heating coils and other components to heat and otherwise prepare the membrane 12, 2522 mounted in the HμB platform 100 for experimentation after incubation. In an example embodiment, the incubator device 2602 includes a sliding lid 2604, a chamber raiser 2606, a slide insert 2608, and a frame 2610. The slide insert 2608 includes at least one slot 2612 to retain the at least one HμB platform 100 for incubation.

As shown in FIG. 27, an alternative embodiment of the system 200 in FIGS. 12-13 has the HμB platform 100 positioned in the incubation device or chamber 2602 placed in a microscope slide insert, such as the product associated with the product code 2×GS-M obtained from Okolab, which fits inside of the frame 2610, such as the product associated with the product code H201-K-FRAME obtained from Okolab. As shown in FIGS. 12-13 and 27, the incubation device or chamber 2062 is sized, configured, and dimensioned to removably sit on top of the stage of an inverted microscope, such as the product associated with the product code DMi8 from Leica Technologies.

In one embodiment, the incubator device or chamber 2602 has 2 mm wide orifices that allow tubing to pass through, so that syringe pumps, such as the syringe pumps 2536 such as shown in FIG. 25, can drive fluid flow into the HμB platform 100 from outside the incubator device or chamber 2602. Two programmable syringe pumps 2536, such as the product associated with the product code NE-1600 obtained from SyringePump, were configured to pump media to the channels 132, 134 of the HμB platform 100 at a rate of, for example, 3 μL/min. in the top channel 132, and a rate of, for example, 1 μL/min. in the bottom channel 132, as shown in FIG. 27. The leads 102, 104 extending out of the electrodes 130, 124, respectively, as shown in FIG. 21B, were clamped and connected, for example, by alligator clamps to an impedance analyzer 2538 such as shown in FIG. 25. For example, the impedance analyzer 2538 is the product associated with the product code IM3570 obtained from HIOKI, to measure the impedance of the cell monolayers 2534 over time. In one embodiment, a MATLAB-based program, script, or code was developed to be executed on a hardware-based computer as shown in FIG. 27, which is used to automate the measurement of impedance in the HμB platform 100, for example, every hour for five days.

The impedance |Z| of DPBS and of the media, such as a combination of DMEM+10% fetal bovine serum (FBS), was measured to ascertain the sensitivity of the HμB platform 100 to capture differences in |Z| for different fluids. Either DPBS or media was introduced in the HμB platform 100 with flow rates of 3 μL/min and 1 μL/min for the channels 132, 134, respectively. Upon |Z| characterization and the establishment of baselines, Muller glia (MG) were added to the top of the membrane 12, 2522 positioned in new HμB platform 100, to measure their impedance for a total of, for example, nine days.

The impedance |Z| of cell barriers was measured via changes in voltage across the porous membrane 12, 2522 using impedance analyzer 2538 is the product associated with the product code IM3570 obtained from HIOKI. Impedance measurements were recorded in a frequency range from 1 Hz to 1 MHz, for example, every hour for four days. The impedance analyzer 2538 was connected to a computer and controlled via a program, script, or code using MATLAB. Using a MATLAB script, the impedance recording was automated, ensuring a timely and accurate recording over the length of the experiment. Based on frequency stability and a discernable change in impedance over time for both cell types MG and ECs, a frequency of 1 MHz was chosen to depict changes in impedance over time. Before starting any impedance recording, a temperature in the chamber was set at 37° C., CO₂ levels at 5%, and fluid flow was at steady state in the HμB platform 100. Additionally, an impedance sweep was performed at least once before starting the automated impedance recording to ensure that there was no electrical interference or noise in the readings.

The barrier permeability was assessed by measuring the rate of convective diffusion of fluorescent dextran of 70 kDa, such as the product associated with product code D1823 obtained from ThermoFisher, across the top and bottom of the porous membrane 12, 2522 over time. Dextran of 70 kDa was reconstituted in DMEM to a concentration of 50 g/mL in a 30 mL syringe, which was connected to a syringe pump 2536 to induce fluid flow on the top side of the membrane 12, 2522 at a rate of 3 μL/min. In addition, a separate 30 mL syringe with unaltered DMEM was placed in a separate syringe pump 2536 to induce fluid flow to the bottom side of the membrane 12, 2522 at a rate of 1 μL/min. Samples of 120 μL of volume from the collecting reservoirs, from the outlets 110, 112 were taken at time intervals of 1 hr, 3 hr, 6 hr, 12 hr, 24 hr, 48 hr, 72 hr, 96 hr, and 120 hrs. A concentration curve was developed, and samples measured for fluorescent intensity using a microplate reader, such as the product associated with the product code SpectraMax M2 obtained from Molecular Devices, with an excitation/emission wavelength ratio of 494/515. The intensity values corresponding to one outlet of all groups were combined, while three replicates of the other outlet were used for each experimental group. Values were normalized to the highest concentration value.

In the systems 200, 2700 shown in FIGS. 12-13 and 27, respectively, an epifluorescence microscope, such as the product associated with the product code DMi8 obtained from Leica, with a cooled CCD camera, such as the product associated with the product code DFC7000 GT obtained from Leica, and microscope software such as software obtained from LAS X Science, were used to capture images in both brightfield and fluorescence via 10× or 20× objective. The navigator function of the LAS X software captured high resolution brightfield images at 10× and stitched them together in a single image, capturing the entire membrane region. Images were processed in ImageJ of the National Institutes of Health (NIH) with respective scalebars. A brightfield upright microscope, such as the product associate with the product code Axio Zoom.V16 obtained from ZEISS, was used to take zoomed-in pictures of a cross-section of the HμB platform 100 using an Axiocam 503 mono camera. Images were manually stitched into a bigger image using Adobe Photoshop.

Two-way analysis of variance (ANOVA) was used to analyze statistical significance among the experimental groups for the impedance and permeability studies in this work. A post-hoc Tukey algorithm was performed to identify the level of statistical significance among the groups. Each experimenter included at least three replicates per experimental condition. Statistical significance is denoted by symbols: *, where p<0.05=* or †, p<0.01=** or ††, p<0.001=***, p<0.0001=****, n.s.=not statistically significant. All statistical tests were performed using a software product associated with the product code Prism 10 obtained from GraphPad.

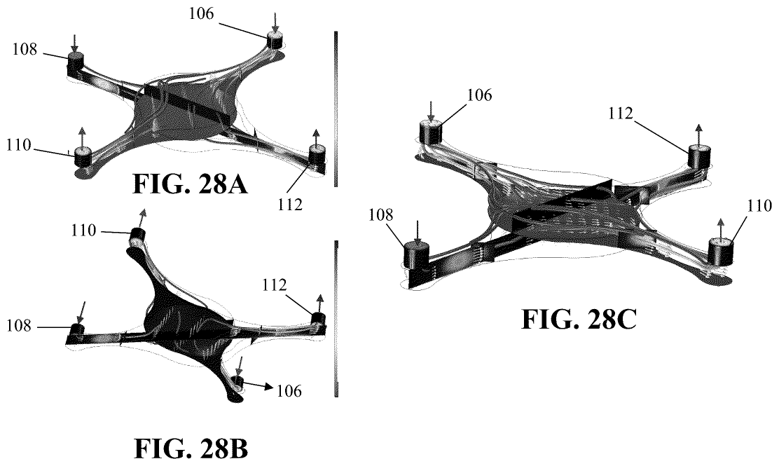

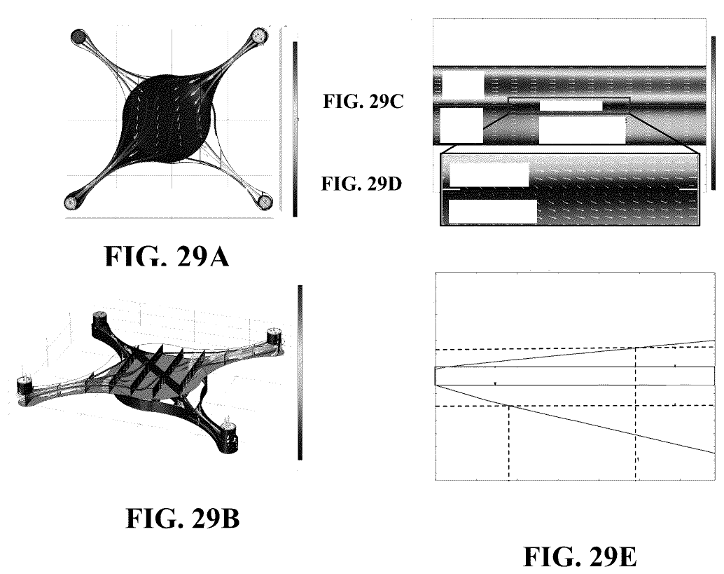

As shown in FIGS. 28A-28C, fluid dynamics simulations demonstrated a uniform flow along the channels 132, 134 and the porous membrane 12. 2522. The highest velocity was displayed in the channels 132, 134 with a range of 5.0×10⁻⁶ m/s to 20.0×10⁻⁶ m/s, where the lowest velocity was found close to the walls, and the highest velocity was found in the center of the channels 132, 134, as per the corollary of a fully developed Poiseuille's flow. The overall lowest velocity of a range of 0.1×10-6 m/s to 5.0×10-6 m/s was found along the porous membrane 12. 2522, as the larger geometry decreased the pressure exerted on the fluid; thus, decreasing the velocity. FIGS. 28A-28C show the magnitude velocity of fluid flow inside the HμB platform 100. The top view in FIG. 28A shows curved or string patterns that correspond to streamlines of fluid flow, which travel along the porous membrane 12, 2522 in a uniform manner, while the bottom view in FIG. 28B also displays the percolating fluid flow across the porous membrane 12, 2522 and exiting through the outlets 110, 112. Yellow arrow field lines in FIGS. 21A-21C show the direction of the fluid flow moving from the inlets 106, 108 to the outlets 110, 112 crossing along and across the porous membrane 12, 2522, as shown in the side view of the simulation in FIG. 21C, as well as in FIG. 29A.

As shown in FIG. 29B, a 2D flow velocity simulation displays the velocity magnitude along and across the porous membrane 12, 2522. The velocity of the fluid close to the membrane 12, 2522 significantly decreased due to friction and an increased vertical velocity component, as fluid flow started percolating the membrane 12, 2522. The fluid velocity that cells experienced on the top channel 132 was approximately 5.8 μm/s at 10 μm height from the membrane 12, 2522, which is within the height range reached by cells cultured in transwells, as shown in FIG. 29B, while cells on the bottom side of the membrane 12, 2522 would experience an average velocity of 2.8 μm/s. These flow velocities correspond to flow rates of 2.7 μL/min on the top side of the membrane 12, 2522, and 1.3 μL/min on the bottom side of the membrane 12, 2522, having an approximate deviation of 0.3 μL/min from the intended flow rate.

FIG. 29C illustrates a two-dimensional graphical representation of a side cross-sectional view of the HμB platform 100 with velocity magnitudes of the fluid flows in the channels 132, 134 and across the membrane 12, 2522. FIG. 29D illustrates an enlarged portion of the graphical representation of the membrane 12, 2522 in FIG. 29C. FIG. 29E illustrates a graph of the velocity magnitudes of the fluid flows in FIG. 29C.

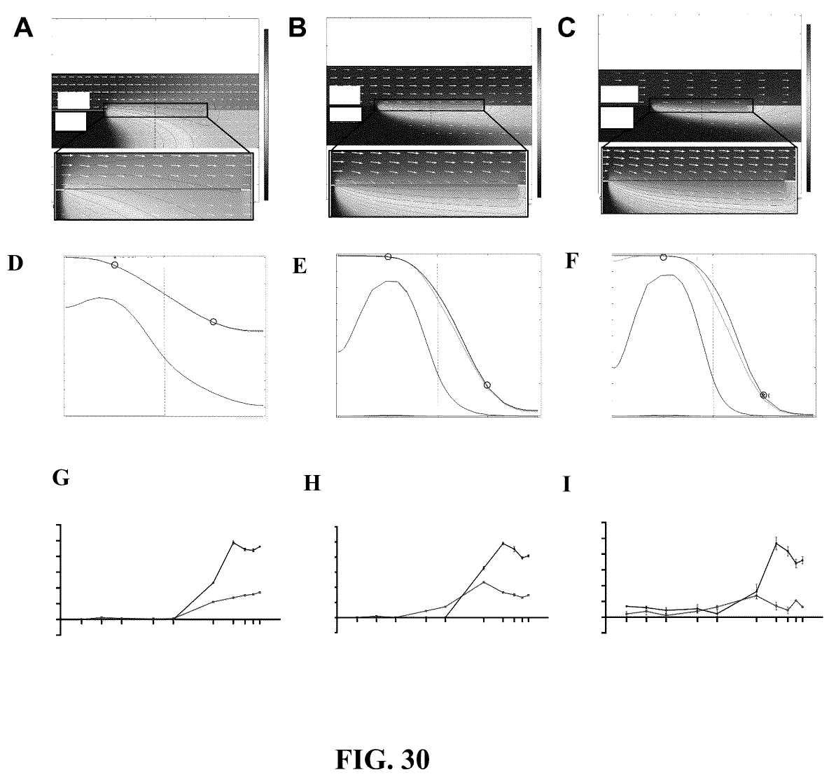

FIGS. 30A-30C illustrate mass transport concentration gradients across the channels 132, 134 in the HμB platform 100 for Rhodamine B, 70 kDa dextran, and 150 kDa dextran, respectively. FIGS. 30D-30F illustrate graphs of concentration gradients across the channels 132, 134 over time in the HμB platform 100 for Rhodamine B, 70 kDa dextran, and 150 kDa dextran, respectively. FIGS. 30G-30I illustrate graphs of normalized concentration of solutes over time at the outlets 110, 112 of the HμB platform 100 for Rhodamine B, 70 kDa dextran, and 150 kDa dextran, respectively.

Mass transport simulations showed that the three tracers, for example, Rhodamine B, 70 kDa dextran, and 150 kDa dextran, had different concentration gradient profiles, as to be expected due to their varying diffusion coefficients. Rhodamine B reached a steady state as soon as 3 hours, while both dextran at 70 kDa and 150 kDa reached steady state at 6 hours. Although both dextrans reach a steady state at 6 hours, the variation on the concentration gradient over time differs. The concentration gradient of the 150 kDa dextran changed at a slower rate than the one of 70 kDa dextran, as its diffusion coefficient is smaller or slower. This is particularly evident in FIGS. 30E-30F between the 1 hr time point (teal color line) and the 5 days line (86,400 s), as the 150 kDa dextran shows a greater change in the rate of concentration, with a gap between these the 1 hr and 5 day line time-points.

The percentage error between the computational and experimental data was calculated for all three tracers: Rhodamine B, 70 kDa dextran, and 150 kDa dextran. Concentration values from the simulation and experimentally obtained for the fifth daytime-point of the experiment demonstrated wide variation. Rhodamine B displayed 8.2% and 35.9% percent error between the computational and experimental data for the top channel 132 and the bottom channel 134, respectively. 70 kDa dextran displayed 16.3% and 66.7% percent error between the computational and experimental data for the top channel 132 and the bottom channel 134, respectively. 150 kDa dextran displayed 26.5% and 4% percent error between the computational and experimental data for the top channel 132 and the bottom channel 134, respectively. FIGS. 30A-30I show the computational and experimental data for the concentration change for Rhodamine B, 70 kDa dextran, and 150 kDa dextran, respectively.

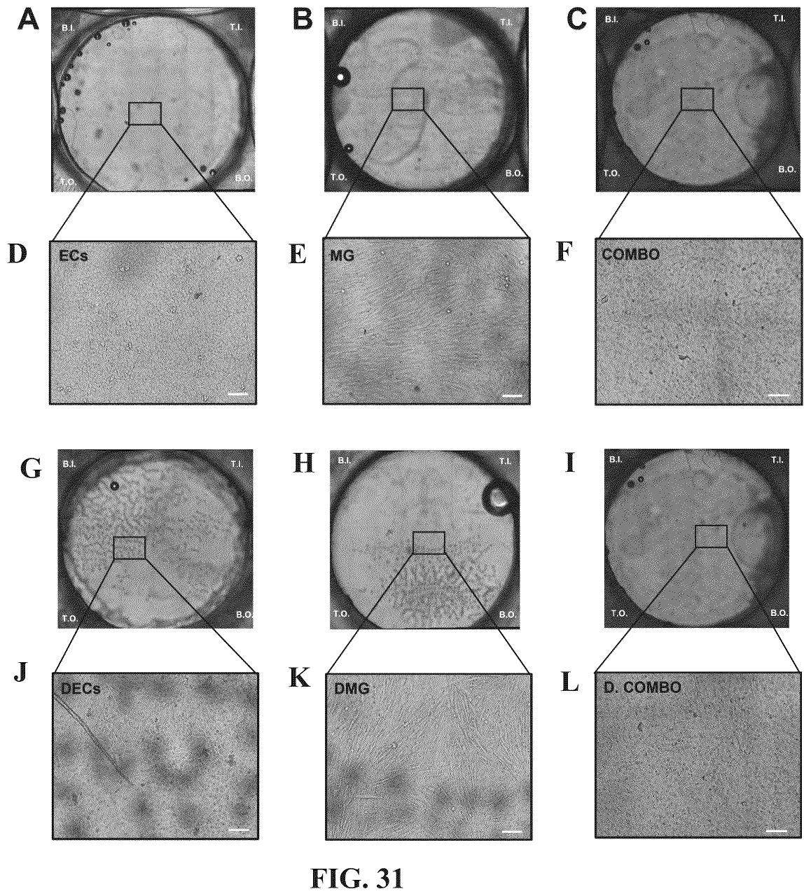

FIGS. 31A-31C illustrate brightfield images of confluent cell barriers under flow for endothelial cells (ECs), Muller glia (MG), and a combination of ECs and MGs, respectively. FIGS. 31D-31F illustrate enlarged portions of the brightfield images of confluent cell barriers under flow shown in FIGS. 31A-31C, respectively. Endothelial cells (ECs), diabetic endothelial cells (DECs), Muller Glia (MG), and diabetic Muller glia (DMG) were seeded individually or in combination (COMBO) and diabetic COMBO (D. COMBO) in the HμB platform 100 as shown in FIGS. 31A-31L. After 48 hr, all cells had formed confluent monolayers and were exposed to fluid flow. Cell barriers were placed inside the incubator device or chamber 2062 on top of the microscope, as shown in FIGS. 12-13 and 27, and exposed to fluid flow for 1 hr before taking pictures in order to allow flow to remove any debris or dead cells. Images of cell barrier shown in FIGS. 31A-31L were collected over a period of 2-3 months during the impedance studies, preceding the permeability studies.

The images in FIGS. 31A-31L show that all of the cells formed confluent cell barriers on the porous membrane 12, 2523. ECs and DECs adopted a hexagonal morphology, and no visible gaps were observed in either cell barrier. However, a greater number of cellular debris was observed in DECs compared to ECs. MG and DMG displayed a fusiform shape, which seemed to be aligned with the direction of flow. This pattern was mostly preserved in the MG group, while the DMG showed an aligned organization of cells, but the pattern was changed in a radius of every 10 cells. COMBO and D. COMBO displayed a combination of the morphology observed in the monocultures of ECs and MG with minimal cell debris compared to the monolayers. The individual cell types were hard to identify in the COMBO and D. COMBO conditions, as cells were only divided by a 12 μm transparent membrane. Overall, all groups displayed healthy and confluent cell barriers, which were used for experiments.

The dark-dotted patterns observed in the images of DECs and DMG in FIGS. 31A-31L are shadows cast from micro bubbles trapped in the glue layer that connected the ITO film and the top electrode layer 124. Due to the cost of materials and the bubbles to be ubiquitously present in this batch of devices, due to the time required for cells to form a barrier, as well as due to cell culture availability, the HμB platforms 100 having such characteristics were used for the experiments. Likewise, it must be made clear that the microbubbles did not interfere with the cell barriers or fluid flow in the HμB platform 100, and the microbubbles are merely an imaging obstruction. The diameter of the top cell barrier is 0.78 cm, while the white scale bar is 100 m.

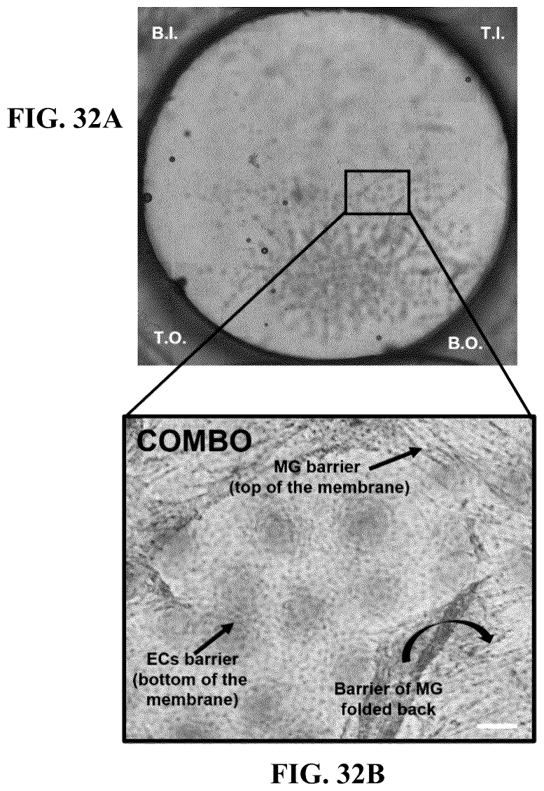

Additionally, as a reference, FIGS. 32A-32B show a COMBO group that experienced an increased shear stress on the bottom channel, in which MG are seeded. The brightfield images of FIGS. 32A-32B are images of a confluent COMBO cell barrier with ECs on the top side of the porous membrane 12, 2522 and MG on the bottom side of the membrane 12, 2522. A flow rate of 100 μL/min instead of 1 μL/min was induced on the bottom channel 134 that interfaces with the MG cell barrier. The shear rate disrupted the MG cell barrier, and caused the MG cell barrier to fold onto itself. The center of the zoomed image in FIG. 32B displays the ECs barrier intact on the other side of the porous membrane 12, 2522, while the MG cell barrier has folded back due to shear stress. The diameter of the top cell barrier is 0.78 cm, while the white scale bar is 100 m. The fluid flow corresponded to 100 μL/min, which was a manual error. The HμB platform 100 in this experiment was discarded, but imaging was performed for future reference on the effects of high shear on cell barriers.

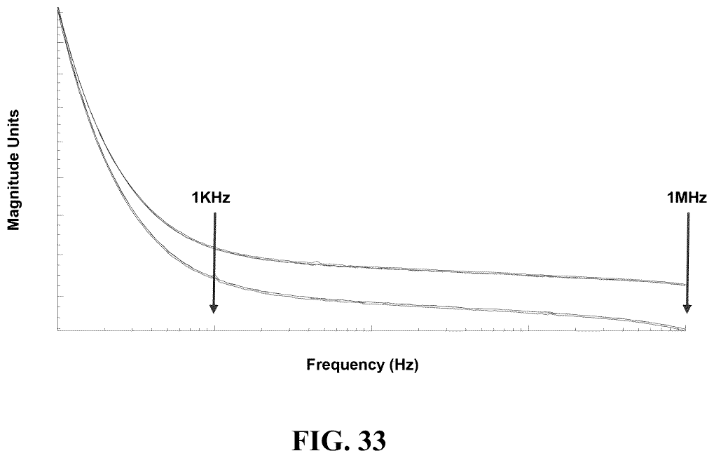

FIG. 33 illustrates a graph of the impedance of the fluids in the HμB platform 100. The impedance of DPBS (PBS) and media (DMEM+FBS) over a 1 Hz to 1 MHz frequency range were measured. Impedance of DPBS and media demonstrated greater sensitivity of the HμB platform 100 to discern |Z| changes from different fluids. FIG. 33 shows that DPBS displayed a higher impedance than the media group over the frequency spectrum of 1 Hz to 1 MHz. The small variation among the multiple recordings for each group also depicts the high accuracy of the system to record impedance.

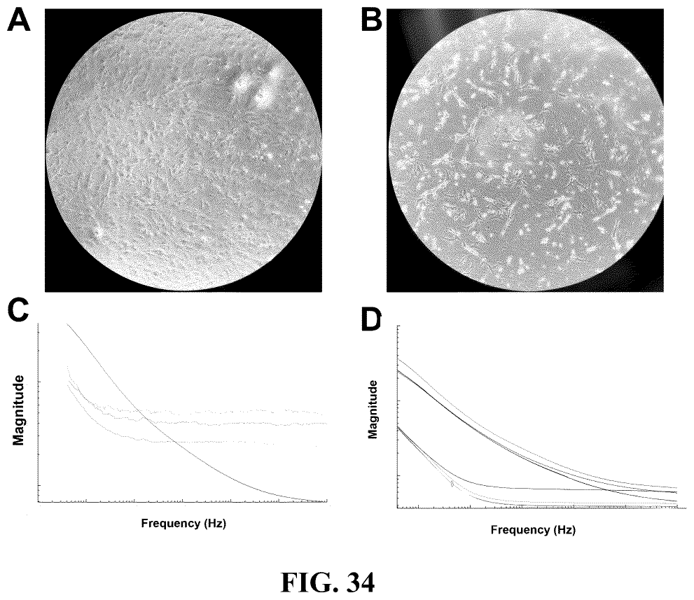

As shown in FIGS. 34A-34D, the impedance of MG was also recorded in two different HμB platforms 100. FIG. 34A illustrates a brightfield image of a healthy confluent cell barrier post-seeding. FIG. 34B illustrates a brightfield image of an unhealthy confluent cell barrier post-seeding. FIG. 34C illustrates a graph of an impedance comparison of the healthy cell barrier from FIG. 34A vs. the unhealthy cell barrier from FIG. 34B. FIG. 34D illustrates a graph of impedance over time of the healthy cell barrier from FIG. 34A. with a first time recording at 3 hrs and a last recording at 9 days. One of the HμB platforms 100 showed significant cell death, as shown in FIG. 34B, while the other HμB platform 100 displayed a fully confluent healthy barrier as shown in FIG. 34A after 24 hrs. In FIG. 34C, the impedance of the healthy barrier showed a stable and smooth magnitude pattern decreasing at a steady rate as frequency increased, as indicated by the purple lines. In FIG. 34D, the impedance of the cells with significant cell death did not change over the frequency spectrum, while the impedance showed minor fluctuations in the entire frequency range. The three iterations of the impedance recording for the “unhealthy” barriers displayed large differences in the magnitude spectrum and no reliable reading was obtained.

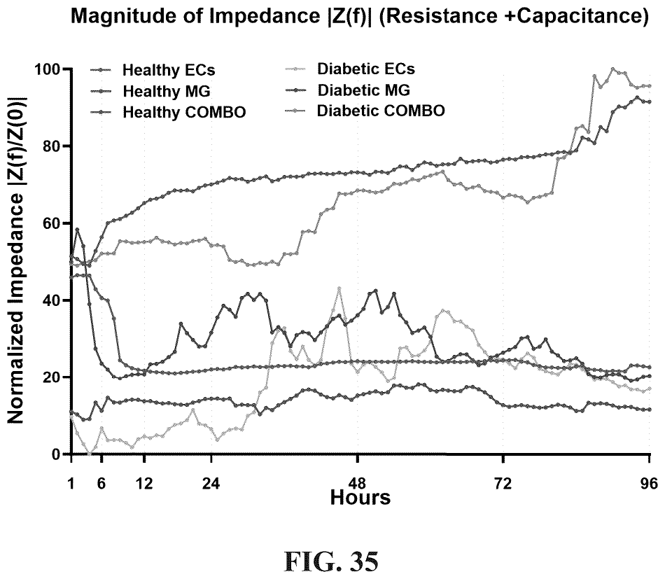

FIG. 35 illustrates a graph of impedance of healthy and diabetic cell barriers in the HμB platform 100 over time. After the healthy barrier was demonstrated a steady magnitude profile, the HμB platform 100 was maintained under flow for 9 days, with intervals of 3 days replacing the media in the syringe pumps 2536 in FIG. 25. After 9 days, the impedance profile for the MG barrier did not change and minimal cell death was observed. The magnitude seemed to decrease by 1 MHz over the period of 1 day and 9 days. Based on the greater differences in the impedance change over time, and so having greater sensitivity, the value of 1MH was selected as the frequency to represent the changes in impedance over time in the impedance studies for all cell barriers.

Healthy cell barriers demonstrated a stable impedance profile, while diabetic cell barriers showed a fluctuating pattern over the course of the study (4 days). Data for COMBO displayed the highest impedance over the first 3.5 days, then the data for COMBO was overtaken by the diabetic COMBO group. The COMBO impedance profile was steady during the first 3.5 days, but then the COMBO impedance increased by ˜30% during the last 0.5 days. The diabetic COMBO displayed a changing impedance profile in a step-like fashion, increasing at the 12 hr, 36 hr, and 82 hr time points, while remaining in a constant impedance range during these intervals. At the 82 hr, the impedance of the diabetic COMBO significantly increased for the next 10 hrs and had overtaken the impedance of the COMBO group by as much as 25%. The impedance of an ECs barrier displayed a decrease during the first 9 hrs, and then the profile stabilized and remained steady throughout the entire study. DECs depicted a highly fluctuating impedance with minor changes during the first 24 hrs, and then significantly increased and had overtaken the impedance of ECs barriers at the 34 hr time point. But then the impedance of the DECs slowly stabilized towards the 72 hr of the study, and slowly dropped below the impedance of the ECs barriers from the 87 hr until the end of the study.

MG cell barriers had a consistent and steady impedance profile in a manner similar to the ECs group, but at a lower impedance. MG had minor impedance fluctuations over the course of the study with small increases at the 30 hr and 85 hr time point. Similar to the DECs group, DMG barriers displayed large fluctuations in their impedance profile with a higher impedance value than the ones of MG, ECs, and DECs groups. The impedance of DMG barriers experienced a significant drop after 3 hrs, only stabilized after 9 hrs, and then increased in an oscillating fashion until the 72 hr time point to remain stable until the end of the study. The impedance values were normalized to the lowest value of zero and the highest value of one-hundred, and the average of each group replicates are plotted in FIG. 35.

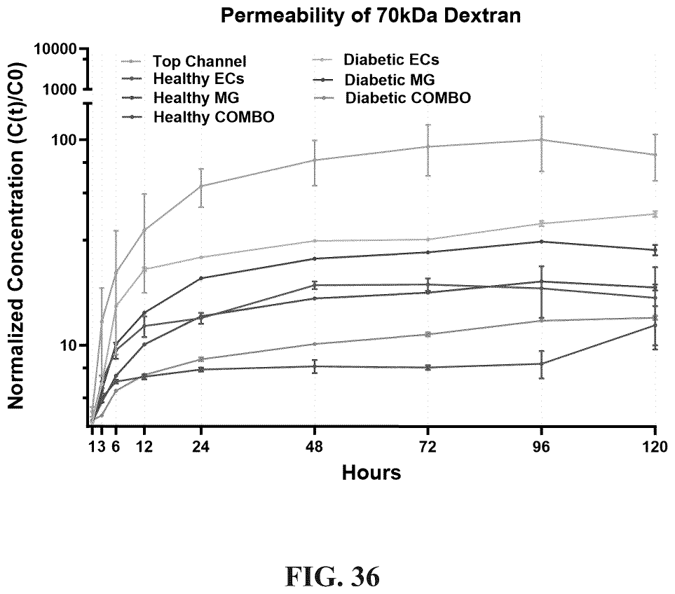

FIG. 36 illustrates a graph of permeability of cell barriers in the HμB platform 100 over time. Normalized permeability of healthy and diabetic cell barriers to 70 kDa dextran are displayed in FIG. 36 over time. Samples of media that exited through the top channel 132 were evaluated via fluorescence spectrometry, averaged among all groups, and plotted as shown by the gray line in FIG. 36. Samples of media exiting via the bottom channel 134 were collected from all groups and their fluorescence assessed at each time point. Normalization was performed with respect to the highest and lowest fluorescence value. The permeability of cell barriers was correlated to the convective diffusion of 70 kDa dextran over time. The permeability profiles for all barrier groups varied with the lowest permeability displayed by the COMBO barrier and the highest permeability by the DECs group. All of the groups demonstrated a steady increase in concentration of dextran across the cell barriers over time. ECs barriers were less permeable over time than DECs, in a manner similar to MG barriers, and were less permeable with respect to DMG. Interestingly, the permeability of ECs and MG barriers was similar all throughout the 5-day study. Although the permeability profiles of DMG and DECs also remained close, there was a bigger difference between these two barriers, in which DECs barriers were more permeable to 70 kDa dextran than DMG barriers. COMBO barriers were more permeable than diabetic COMBO barriers during the first 12 hrs of the study, and then diabetic COMBO barriers steadily became more permeable than the COMBO group over time. Furthermore, at the 96 hr time point, COMBO barriers displayed a sudden increased in permeability until the 120 hr time point, reaching a similar permeability value than the diabetic COMBO group, as shown in FIG. 36.

As disclosed herein, the development of the multi-layered microfluidic system 200, 2700 on a bench-top setting using an easily fabricated HμB platform 100 sets a precedent for biological sciences to embrace the use of this highly customizable technology to leverage scientific research. In particular, the HμB platform 100 has been demonstrated to be precise, highly replicable, and easy to assemble with minor training, effectively migrating the clean room to the makerspace. FIGS. 3-4, 10-11, and 21A-21C show the different layers that constitute the assembled HμB platform 100 with a total material cost of approximately USD $3.20 per device. The automated laser-cutting of the acrylic sheets maintained the dimensions of the HμB platform 100 to be invariable with an approximate laser-cutting rate of 20 mins to make 20 or more of such HμB platform 100. The cross-sectional image from FIG. 11 shows that, despite that the HμB platform 100 was compressed by the heated press 2410 shown in FIG. 24E, the channels 132, 134 maintain their structure, and the porous membrane 12, 2522 remains fixed between the two channels 132, 134. In addition, the HμB platform 100 was able to resist autoclaving at 15 psi with a temperature of 121° C. for over 15 minutes, which could allow the HμB platform 100 to be reusable.

Fluids in the HμB platform 100 flowed along the top and bottom of the porous membrane 12, 2522, and exited via the outlets 110, 112 without leaks or visible obstruction of fluid flow. Furthermore, fluid velocity simulations demonstrated that the fluid flow is uniform throughout the HμB platform 100, achieving a laminar flow profile as depicted by at least FIGS. 29A-29B. Such fluid flows are representative in the retinal microvasculature, where laminar flow is predominant. Other studies have illustrated that the geometry of the blood vessel plays a fundamental role in the flow profile of blood, where blood vessel bifurcation, as well as the presence of stenosis, can lead to turbulence in the flow. Nonetheless, the HμB platform 100 is able to recapitulate the flow profile found in microcapillaries within a physiological range of flow rates. Additionally, the fluid flow simulations show that the flow velocity was within the expected range at the 10 μm height, which is the height for monolayers on a porous membrane 12, 2522. Such flow velocity is particularly important as fluid flow will provide nutrients to the cells and remove waste without exerting high degree shear of shear that may compromise the cell barrier integrity.

The convective-diffusion transport of the different molecular weight (M.W.) of dextran and Rhodamine B provided insight into the ability of solutes to cross the porous membrane 12, 2522 over time. Lower molecular weight solutes such as Rhodamine B crossed the porous membrane 12, 2522 and established a concentration gradient that reached steady state at a shorter time, while covering a larger area when compared to the dextrans, as shown in FIGS. 30A-30I. Higher molecular weight solutes such as 70 kDa dextran and 150 kDa dextran also crossed the porous membrane 12, 2522 and established a concentration gradient that reached steady state about 6 hrs later. These simulations provide insight on how other natural solutes of similar molecular weight (i.e. albumin with a M.W. of 69 kDa) or therapeutics can cross cell-barriers over time. Although the experimental values of solute concentration did not perfectly match the expected concentration values from the simulation, the experimental values demonstrated that solutes of different molecular sizes are able to percolate the porous membrane 12, 2522, as well as showing that there is no backflow since the concentration of the top channel 132 was always higher than the bottom channel 134. Some possible explanations for the discrepancy in the obtained and expected values may be attributed to the imperfection of the computational model to account for parameters such as the lipophilic characteristics of the porous membrane 12, 2522, early release of solute media during the experimental set-up due to human error, and small hydrostatic changes between the outlet connectors and the tubing over the course of 5 days that might have slowed down the exit of the fluid.

The HμB platform 100 has been demonstrated to be an optimal cell culture platform, as both ECs and MG were able to form confluent cell barriers on either side of the porous membrane 12, 2522. FIGS. 31A-31L show that cell confluency, proliferation, and morphology can be measured over time, which is an important metric of cellular behavior. The morphology of ECs and MG also varied due to their cell size and their ability to form junctions. ECs form tight junctions, which allow the ECs to create tight barriers with minimal free space between them, while MG only connect via gap junctions when their cell population is confluent. Additionally, MG are known to increase their secretion of extracellular matrix products when becoming hypertrophic, which may create a barrier between cells. The images in FIGS. 31A-31L also depict a flow-dependent orientation in MG barriers, in which the soma of the MG barriers seems to be arranged in a uniform direction, agreeing with the morphology documents by other groups. In addition, FIGS. 32A-32B also demonstrate the sensitivity of MG, since significantly higher shear, due to increased flow velocity, disrupted the MG monolayer. However, at given flow rates of 3 μL/min and 1 μL/min, the cell barriers grew to confluency with a healthy phenotype.

The electrodes 124, 130 of the HμB platform 100 showed high impedance sensitivity to the solutes in the medium, as the difference in impedance values between DPBS and complete media, as shown in FIG. 33. In addition, disparities in cell barrier health were clearly outlined by the changes in impedance between healthy and unhealthy MG, illustrated in FIGS. 34A-34D. The fluctuation of the impedance values in the unhealthy group with respect to the healthy group of MG monolayers showed that changes in cellular behavior such as cell density, cell shape, metabolic activity, and cell viability are metrics that can be measured via impedance, which makes the HμB platform 100 a great tool to assess cellular behavior in real time. This phenomenon was also observed in the impedance profiles of healthy and diabetic cell barriers at the 1 kHz frequency shown in FIG. 35. The diabetic cell barriers demonstrated great impedance fluctuation over time when compared to their healthy counterparts. However, the diabetic COMBO group depicted a more stable impedance profile when ECs and MG are seeded on opposite sides of the membrane 12, 2522, and possible cellular communication via paracrine signaling between the two cell types may encourage barrier stability and integrity. By contrast, healthy cell barriers demonstrated a uniform impedance profile over time, with the highest impedance led by the COMBO group and the lowest impedance by the MG group.

The highest impedance by the COMBO and diabetic COMBOs was not surprising, as highest cell density leads to higher impedance with respect to the monolayer groups. Healthy ECs barriers displayed the most uniform and stable impedance profile among all groups with no significant changes over time, which alludes to the ability of ECs to form tight cell barriers and maintain the unchanged structure over time. On the other hand, diabetic ECs displayed the highest fluctuations among all groups during the first 72 hrs, then stabilizing afterwards. Although the cell barriers of ECs remained confluent and did not display any observable changes in morphology or cell density, changes in cell impedance shed light on their inability to form a stable integral barrier. Changes in barrier homeostasis and ability to form functional tight junctions could explain the differences in impedance profiles between healthy ECs and diabetic ECs. Similarly, differences in barrier impedance in MG and diabetic MG might be due to changes in their cellular dynamics. Yet, higher impedance in the diabetic MG group might be attributed to acquired hypertrophy, which increases cellular density in glial cells and allow them to build thicken extracellular matrices due to an increasing production of collagen IV.

Differences in cell barrier permeability displayed a direct correlation with the results from the impedance study. The healthy and diabetic COMBO groups, which demonstrated the highest barrier impedance, also displayed the lowest permeability, as shown in FIG. 36. The contrary phenomenon was observed in DECs and DMG cell barriers, which demonstrated the lowest impedance profile with the greatest fluctuations. These results agree with what has been illustrated in other cell barrier studies in vitro, where high glucose leads to a decrease in barrier resistance, where hyperpermeability is linked to cell junction disfunction, resulting in lower barrier integrity. Furthermore, the permeability rate to 70 kDa dextran is maintained over time which plateaus after approximately 48 rs for most cell barrier groups. This phenomenon can be attributed to the formation of a concentration gradient across the cell barrier at steady state conditions, as illustrated in FIGS. 30B, 30E, and 30H, in which 70 kDa dextran reached stay state across the porous membrane 12, 2522 after 6 hrs (no cells). Once steady state conditions are achieved across the cell barrier, the rate of solute transport is maintained constant, and partially restricted, as shown by the experimental results using the HμB platform 100.

Overall, the experimental results using the HμB platform 100 describe the development of a novel, low-cost, and easy to manufacture microfluidic system 200, 2700 that enables the culture of cell barriers in situ. These system 200, 2700 are capable of recording changes in cell-based impedance and allowing imaging in real-time under fluid flow conditions. Using these system 200, 2700, physiological differences were identified in integrity and permeability of healthy and diabetic cell barriers of ECs and MG. The experimental results showed that diabetic cell barriers develop hyperpermeability in response to hyperglycemia and the presence of advanced glycation end-products. In addition, diabetic cell barriers displayed an unstable impedance profile with large variation in impedance values, while healthy cell barriers displayed a stable impedance profile over time.

The HμB platform 100, the systems 200, 2700, and the method 30 are able to examine the integrity of biobarriers 12, such as blood barriers. The blood barriers can be an inner blood-retinal barrier (IBRB) as well as neurovascular barriers along different parts of the brain and spinal cord, the placental blood barrier, and a blood testing barrier. The HμB platform 100, the system 200, and the method 300 enable the study of molecular transport across multiple dynamic monolayers using distinct flow conditions for each monolayer. The HμB platform 100, the system 200, and the method 300 facilitate measurement of electrical conductivity, resistivity, and permeability across individual and combined monolayers over time in response to natural bio-compounds, such as glucose, as well as commercial pharmacology, such as bevacizumab/AVASTIN.

In addition, the HμB platform 100 can include electrodes composed of plastic, which makes the overall device inexpensive to manufacture and easy to assemble. The electrodes can be composed of, for example, acrylic sheets bonded to a (conductive) film of indium-tin-oxide, which allows the electrodes to be conductive on the side of the film and to work as electrodes. Alternatively, the film can be composed of any known film material. The side of the film faces inward towards the biobarrier 12 or membrane. In another embodiment, the acrylic sheet and the film forming the electrodes can be transparent. It will be appreciated that indium tin-oxide is one exemplary conductive film that can be used and other conductive films can be used in its place on the plastic electrode.

It will also be understood that the HμB platform 100 is a self-contained portable structure that can easily be connected to fluid sources and is configured to permit imaging of the biobarrier 12 during performance of test conditions. In addition, the design of the HμB platform 100 is capable of reaching ultra-low flow rate (e.g., in the bottom microchannel). For example, an ultra-low flow rate can be defined to be less than 5 μL/min and in some embodiments, less than 3 μL/min and yet in other embodiments, less than 1 μL/min. The HμB platform 100 can thus be incorporated into a microfluidic system that replicates the close cell proximity of any tissue barrier of the body under physiological flow conditions.

It is to be further understood that like or similar numerals in the drawings represent like or similar elements through the several figures, and that not all components or steps described and illustrated with reference to the figures are required for all embodiments or arrangements.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “contains”, “containing”, “includes”, “including,” “comprises”, and/or “comprising,” and variations thereof, when used in this specification, specify the presence of stated features, integers, steps, operations, elements, and/or components, but do not preclude the presence or addition of one or more other features, integers, steps, operations, elements, components, and/or groups thereof.

Terms of orientation are used herein merely for purposes of convention and referencing and are not to be construed as limiting. However, it is recognized these terms could be used with reference to an operator or user. Accordingly, no limitations are implied or to be inferred. In addition, the use of ordinal numbers (e.g., first, second, third) is for distinction and not counting. For example, the use of “third” does not imply there is a corresponding “first” or “second.” Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

While the disclosure has described several exemplary embodiments, it will be understood by those skilled in the art that various changes can be made, and equivalents can be substituted for elements thereof, without departing from the spirit and scope of the invention. In addition, many modifications will be appreciated by those skilled in the art to adapt a particular instrument, situation, or material to embodiments of the disclosure without departing from the essential scope thereof. Therefore, it is intended that the invention not be limited to the particular embodiments disclosed, or to the best mode contemplated for carrying out this invention, but that the invention will include all embodiments falling within the scope of the appended claims.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

What is claimed is:

1. A high efficiency microfluidic bio-barrier (HμB) platform comprising:
a first inlet configured to receive a first fluid;
a second inlet configured to receive a second fluid;
a first outlet to discharge the first fluid;
a second outlet to discharge the second fluid;
a first member configured to retain a biobarrier, the first member including a first microchannel in fluid communication with the first inlet to receive the first fluid and to pass the first fluid adjacent to a first face of the biobarrier, the first outlet being in fluid communication with the first microchannel; and
a second member configured to retain the biobarrier, with the biobarrier being disposed between the first member and the second member, the second member including a second microchannel in fluid communication with the second inlet to receive the second fluid and to pass the second fluid adjacent to a second face of the biobarrier that is opposite the first face, the second outlet being in fluid communication with the second microchannel.

2. The high efficiency microfluidic bio-barrier (HμB) platform of claim 1, wherein the first inlet, the second inlet, the first outlet and the second outlet are disposed along a top of the high efficiency microfluidic bio-barrier (HμB) platform.

3. The high efficiency microfluidic bio-barrier (HμB) platform of claim 1, further including at least one frame that includes a window for viewing the biobarrier, the first inlet comprising a first port that projects outwardly from the at least one frame, the second inlet comprising a second port that projects outwardly from the at least one frame, the first outlet comprising a third port that projects outwardly from the at least one frame, and the second outlet comprising a fourth port that projects outwardly from the at least one frame, the at least one frame includes four through apertures that are in fluid communication with the first port, the second port, the third port, and the fourth port, as well as the first microchannel and the second microchannel.

4. The high efficiency microfluidic bio-barrier (HμB) platform of claim 3, wherein the at least one frame comprises a plastic plate.

5. The high efficiency microfluidic bio-barrier (HμB) platform of claim 1, wherein each of the first member and the second member comprises a plate.

6. The high efficiency microfluidic bio-barrier (HμB) platform of claim 1, wherein the first microchannel and the second microchannel define an X shape, with the first microchannel being located above in a different plane than the second microchannel.

7. The high efficiency microfluidic bio-barrier (HμB) platform of claim 1, wherein the first microchannel and the second microchannel are formed parallel to one another.

8. The high efficiency microfluidic bio-barrier (HμB) platform of claim 1, wherein the first member comprises a first plate, the second member comprises a second plate, the first microchannel comprises a void formed in the first member, and the second microchannel comprises a void formed in the second member, with the biobarrier being disposed between the first microchannel of the first plate and the second microchannel of the second plate.

9. The high efficiency microfluidic bio-barrier (HμB) platform of claim 8, wherein each of the first plate and the second plate comprises a clear transparent plastic plate.

10. The high efficiency microfluidic bio-barrier (HμB) platform of claim 1, further including a first electrode with a first conductive lead and a second electrode with a second conductive lead, the first electrode being disposed along a top surface of the first member and the second electrode being disposed along a bottom surface of the second member.

11. The high efficiency microfluidic bio-barrier (HμB) platform of claim 10, wherein the first electrode comprises a first electrode plate formed of a conductive plastic and the second electrode comprises a second electrode plate formed of the conductive plastic.

12. The high efficiency microfluidic bio-barrier (HμB) platform of claim 11, wherein each of the first electrode plate and the second electrode plate is composed of a transparent plastic sheet bonded to a transparent electrically conductive film of indium-tin-oxide.

13. The high efficiency microfluidic bio-barrier (HμB) platform of claim 10, wherein the first conductive lead and the second conductive lead are located at a first end of the high efficiency microfluidic bio-barrier (HμB) platform.

14. The high efficiency microfluidic bio-barrier (HμB) platform of claim 10, wherein the first electrode, the first member, the second member and the second electrode are oriented parallel to one another in a stacked arrangement.

15. The high efficiency microfluidic bio-barrier (HμB) platform of claim 14, wherein each of the first member and the second member comprises a plate formed of a nonconductive material.

16. The high efficiency microfluidic bio-barrier (HμB) platform of claim 14, wherein the second electrode comprises a bottommost layer of the high efficiency microfluidic bio-barrier (HμB) platform.

17. The high efficiency microfluidic bio-barrier (HμB) platform of claim 1, wherein the first microchannel comprises an enlarged center region and the second microchannel comprises an enlarged center region, the first enlarged region and the second enlarged region being aligned with and being located directly above and below the biobarrier, respectively.

18. The high efficiency microfluidic bio-barrier (HμB) platform of claim 1, wherein each of the first inlet, the second inlet, the first outlet and the second outlet comprises a Luer-lock.

19. The high efficiency microfluidic bio-barrier (HμB) platform of claim 1, wherein the first inlet and the second inlet are located side-by-side and the first outlet and the second outlet are located side-by-side with a window defined therebetween, the window configured to permit visual observation of the biobarrier to determine a visual property of the biobarrier.

20. The high efficiency microfluidic bio-barrier (HμB) platform of claim 19, wherein the window is an empty aperture.

21. The high efficiency microfluidic bio-barrier (HμB) platform of claim 1, wherein the biobarrier comprises a porous membrane that supports a cellular structure.

22. A microfluidic system, comprising:
the high efficiency microfluidic bio-barrier (HμB) platform of claim 1;
a first fluid delivery device to deliver the first fluid to the first inlet at a controllable first flow rate; and
a second fluid delivery device to deliver the second fluid to the second inlet at a controllable second flow rate.

23. The system of claim 22, wherein the first fluid is different than the second fluid.

24. The system of claim 22, wherein the first flow rate is different than the second flow rate.

25. The system of claim 24, wherein the first flow rate mimics a capillary flow rate and the second flow rate mimics an interstitial fluid flow rate.

26. The system of claim 22, further comprising:
a solute concentration measuring device configured to measure a concentration of solutes in at least one of the first outlet fluid and the second outlet fluid, thereby measuring a permeability of the biobarrier.

27. The system of claim 22, further comprising a visual observation device adjacent to the high efficiency microfluidic bio-barrier (HμB) platform; and
wherein the first member includes a window adjacent to the biobarrier,
wherein the visual observation device is disposed adjacent to the window, and
wherein the first member is configured to permit visual observation of the biobarrier by the visual observation device through the window to determine a visual property of the biobarrier.

28. The system of claim 27, wherein the window is an empty aperture.

29. The system of claim 27, wherein the visual observation device is selected from the group consisting of a microscope, a lens, and a camera.

30. The system of claim 22, further comprising:
an electrical analyzer configured to determine an electrical property of the biobarrier;
wherein the platform includes a first electrode disposed adjacent to the first microchannel and being electrically conductive,
wherein the first electrode is electrically connected to the electrical analyzer to permit an electrical measurement of the biobarrier to determine the electrical property of the biobarrier.

31. The system of claim 30, wherein the electrical property of the biobarrier is selected from the group consisting of: an electrical capacitance, an electrical impedance, an electrical voltage, and an electrical resistivity.

32. A high efficiency microfluidic bio-barrier (HμB) platform comprising:
a first inlet configured to receive a first fluid;
a second inlet configured to receive a second fluid;
a first outlet to discharge the first fluid;
a second outlet to discharge the second fluid;
a first member configured to retain a biobarrier, the first member including a first microchannel in fluid communication with the first inlet to receive the first fluid and to pass the first fluid adjacent to a first face of the biobarrier, the first outlet being in fluid communication with the first microchannel;
a second member configured to retain the biobarrier, with the biobarrier being disposed between the first member and the second member, the second member including a second microchannel in fluid communication with the second inlet to receive the second fluid and to pass the second fluid adjacent to a second face of the biobarrier that is opposite the first face, the second outlet being in fluid communication with the second microchannel; and
a first electrode with a first conductive lead and a second electrode with a second conductive lead, the first electrode being disposed along a top surface of the first member and the second electrode being disposed along a bottom surface of the second member,
wherein the first electrode comprises a first electrode plate formed of a conductive plastic and the second electrode comprises a second electrode plate formed of the conductive plastic.

33. The high efficiency microfluidic bio-barrier (HμB) platform of claim 32, wherein each of the first electrode plate and the second electrode plate is composed of a transparent plastic sheet bonded to a transparent electrically conductive film.

34. The high efficiency microfluidic bio-barrier (HμB) platform of claim 33, wherein the transparent electrically conductive film is composed of indium tin oxide.

35. The high efficiency microfluidic bio-barrier (HμB) platform of claim 32, further including at least one frame that includes a window for viewing the biobarrier, the first inlet comprising a first port that projects outwardly from the at least one frame, the second inlet comprising a second port that projects outwardly from the at least one frame, the first outlet comprising a third port that projects outwardly from the at least one frame, and the second outlet comprising a fourth port that projects outwardly from the at least one frame, the at least one frame includes four through apertures that are in fluid communication with the first port, the second port, the third port, and the fourth port, as well as the first microchannel and the second microchannel.

36. The high efficiency microfluidic bio-barrier (HμB) platform of claim 35, wherein the at least one frame comprises a plastic plate.

37. The high efficiency microfluidic bio-barrier (HμB) platform of claim 36, wherein the at least one frame comprises a clear transparent plastic plate.
